(19) United States
(12) Patent Application Publication
(10) Pub. No.: US 2023/0072872 A1
(43) Pub. Date: Mar. 9, 2023

(54) System and method of marking cardiac time intervals from the heart valve signals using a Near-Field Communication based patch biosensor

(71) Applicant: AventuSoft, LLC, Boca Raton, FL (US)

(72) Inventors: Arash Andalib, Delray Beach, FL (US); Kaustubh Kale, Delray Beach, FL (US)

(73) Assignee: AventuSoft, LLC, Boca Raton, FL (US)

(21) Appl. No.: 17/983,343

(22) Filed: Nov. 8, 2022

Related U.S. Application Data
(63) Continuation-in-part of application No. 17/982,510, filed on Nov. 7, 2022.
Application No. 17/982,510 is a continuation-in-part of application No. 16/741,740, filed on Jan. 13, 2020, now Pat. No. 11,490,849.
Application No. 16/741,740 is a continuation-in-part of application No. 15/397,138, filed on Jan. 3, 2017, now Pat. No. 10,531,839.

(51) Int. Cl. A61B 5/0205; A61B 5/00; A61B 5/257

(52) U.S. Cl. CPC A61B 5/0205; A61B 5/002; A61B 5/257; A61B 5/7278; A61B 5/08

(57) ABSTRACT

A health sensor system and method can include a wearable non-invasive biosensor for capturing cardiac waveform signals such as electrocardiogram (ECG) signals and composite vibration objects over one or more channels, one or more processors operatively coupled to the wearable non-invasive biosensor, and memory having computer instructions which causes the system to perform certain operations. In some embodiments, the operations can include powering the health sensor system in response to receiving a radio frequency signal using a near field communication protocol, monitoring pulmonary artery pressures based on cardiac time intervals during a period when the health sensor system is powered by the radio frequency signal, performing a heart and lung function assessment based on the monitoring of the pulmonary artery pressures, and presenting the heart and lung function assessment. In some embodiments, the biosensor can be a single NFC patch biosensor.

### CROSS REFERENCE TO RELATED APPLICATIONS

This Application is a Continuation-in-Part of and claims priority through U.S. patent application Ser. No. 17/982,510 filed on Nov. 7, 2022 and a Continuation-in-part through U.S. patent application Ser. No. 16/741,740 filed on Jan. 13, 2020, which claims priority through U.S. patent application Ser. No. 15/397,138 filed on Jan. 3, 2017 which further claims the priority benefit of Provisional Application Nos. 62/274,766, 62/274,761, 62/274,763, 62/274,765, and 62/274,770 each of which were filed on Jan. 4, 2016, the entire disclosure of each are incorporated herein by reference.

### FIELD

The embodiments herein relate generally to cardiopulmonary health monitoring and more particularly to analysis software combined with non-invasive transducers to capture multi-channel vibration signals along with an electrocardiogram signal for the measurement of heart functions and their related diseases.

### BACKGROUND

Heart disease is the leading cause of death accounting for more than one-third (33.6%) of all U.S. deaths. Overall cardiac health can be significantly improved by proper triage. Low invasive and non-invasive ultrasound techniques (e.g. echocardiogram) are standard procedures, but the requirement of expensive devices and skilled operators limit their applicability. The following are the various types of heart disease that can be diagnosed and treated using the separated signal, namely, Coronary artery disease, Heart murmurs and valve abnormalities, Heart failure, Heart rhythm abnormalities (arrhythmias), Vascular disease, congenital heart disease, Cardiac resynchronization and Risk factor modification. A physician can work with patients to perform a comprehensive evaluation and design a personalized plan of care aimed at keeping them healthy.

The cardio pulmonary system which consists of the respiratory components, snoring components, and cardiac components, creates vibrations during each cardiac cycle. The vibrations are the result of the lung sounds, heart sounds, acceleration and deceleration of blood due to abrupt mechanical opening and closing of the heart valves during the cardiac cycle.

### SUMMARY

The exemplary embodiments herein provide a method and system of health monitoring. In one embodiment, a health sensor system includes a wearable non-invasive biosensor for capturing electrocardiogram (ECG) signals and composite vibration objects over one or more channels using a near field communication protocol, one or more processors operatively coupled to the wearable non-invasive biosensor, and memory having computer instructions which when executed by the one or more processors causes the system to perform certain operations. In some embodiments, the operations can include powering the health sensor system in response to receiving a radio frequency signal, monitoring pulmonary artery pressures based on cardiac time intervals during a period when the health sensor system is powered by the radio frequency signal, performing a heart and lung function assessment based on the monitoring of the pulmonary artery pressures, and presenting the heart and lung function assessment.

In some embodiments, the wearable non-invasive biosensor is a patch biosensor. In some embodiments, the wearable non-invasive biosensor is a single near-field communication patch biosensor.

In some embodiments, the cardiac time intervals are measurements of one or more of isovolumic contraction time, aortic opening time, Mitral closing time, Mitral opening time, aortic closing time, and systolic time ratio.

In some embodiments, the wearable non-invasive biosensor uses a 1-lead ECG sensor and a single channel accelerometer or cardiac waveform sensor to provide data supporting frequent usage at a data rate compatible with the near field communication protocol.

In some embodiments, the wearable non-invasive biosensor is a patch sensor that frequently captures no more than 10 seconds of 1-lead ECG data and a single channel accelerometer data (or single channel of cardiac waveform data) to provide data supporting frequent usage at a data rate compatible with the near field communication protocol.

In some embodiments, the wearable non-invasive biosensor is a patch sensor that frequently captures a predetermined time period of data to perform hemodynamic assessment measurements of heart rate, breathing rate, 1-lead ECG data, and 1 channel of heart sound data. In some embodiments, presenting the heart and lung function assessment includes displaying the heart and lung function assessment on a phone in close proximity to the patch sensor. In some embodiments, presenting the heart and lung function assessment includes displaying the heart and lung function assessment on a remote monitoring station or display. In some embodiments, the one or more processors further perform the operation of securely transmitting via a HIPAA-compliant network for remote monitoring of the hemodynamic assessment measurements.

In some embodiments, the wearable non-invasive biosensor is a patch sensor that frequently captures a predetermined time period of data to perform hemodynamic assessment measurements of one or more among Right Ventricular Systolic Pressure (RVSP), mean Pulmonary Artery Pressure (mPAP), Left ventricular end-diastolic pressure (LVEDP), E/e′, Ejection fraction or Left Atrial Volume Index.

In some embodiments, the one or more processors further perform the operation of estimating absolute hemodynamics using cardiac time intervals defined by opening and closing of valves of a heart with respect to a start of a QRS complex as measured by the wearable non-invasive biosensor capturing ECG signals.

In some embodiments, a method of providing a heart and lung function assessment using a wearable non-invasive biosensor can include the steps of capturing electrocardiogram (ECG) signals and composite vibration objects over one or more channels using a near field communication protocol using the non-invasive biosensor in the form of a single near-field communications patch biosensor, powering the non-invasive biosensor in response to receiving a radio frequency signal, monitoring pulmonary artery pressures based on cardiac time intervals during a period when the health sensor system is powered by the radio frequency signal, performing a heart and lung function assessment based on the monitoring of the pulmonary artery pressures, and presenting the heart and lung function assessment.

In some embodiments, the cardiac time intervals are measurements of one or more of isovolumic contraction time, aortic opening time, Mitral closing time, Mitral opening time, aortic closing time, and systolic time ratio.

### DESCRIPTION OF THE DRAWINGS

FIG. 1A illustrates a system for the extraction, identification, marking and display of the heart valve signals in accordance with one embodiment;

FIGS. 1B and 1C illustrate cardio pulmonary signal capture at the chest in accordance with various embodiments;

FIG. 2 is a flowchart of a method practiced by the system in accordance with one embodiment;

FIG. 3 illustrates multichannel signals captured from the sensor array on the chest shown in accordance with one embodiment;

FIG. 4 illustrates a cardiac cycle in relation with Electrocardiogram, acoustic and accelerometer sensors (or cardiac waveform sensors) of the system in accordance with one embodiment;

FIG. 5 illustrates a heart anatomy and schematic representation of the cardiopulmonary sounds in relation to electrocardiogram;

FIGS. 6A, 6B, and 6C illustrate a method and a cardiac time interval measurement in accordance with one embodiment;

FIGS. 7A and 7B illustrate the marking of vibration objects or each valve into individual streams in accordance with one embodiment;

FIGS. 8A, 8B, and 8C illustrate the comparison of M1, T1, A2, and P2 timings and comparison of time calculations using different energy thresholds in accordance with one embodiment;

FIG. 9 is a flowchart of a method practiced by the system in accordance with an embodiment;

FIG. 10 is an illustration of a dictionary of signal-atoms (on a left side) and a weight matrix/loadings (on a right side) given by a sparse coding source separation algorithm in accordance with an embodiment;

FIG. 11 is an illustration of a sparse projection of an activation pattern including PCA projection followed by subspace rotation in accordance with an embodiment;

FIG. 12 is an illustration of a system based on deep learning that learns to generate cardiovascular absolute markers in accordance with an embodiment; and

FIG. 13 is an example of data labels (pronounced dots or green stars if shown in color) for Aortic valve opening given by an automatic labeling algorithm applied to Hemotag single channel data in accordance with an embodiment.

FIG. 14 illustrates a health sensor system a wearable non-invasive biosensor and smartphone in accordance with an embodiment;

FIG. 15 illustrates a non-invasive NFC patch sensor as placed on a patient's sternum in accordance with an embodiment;

FIG. 16 illustrates a non-invasive patch sensor as placed on a patient's sternum in accordance with an embodiment;

FIG. 17 is an illustration of cardiac timing intervals assessed by a tissue Doppler imaging device;

FIG. 18 represents an algorithm for cardiac timing intervals in accordance with an embodiment; and

FIG. 19 represents a block diagram of a health sensor system in accordance with the embodiments.

### DETAILED DESCRIPTION

The exemplary embodiments may be further understood with reference to the following description and the appended drawings, wherein like elements are referred to with the same reference numerals. The exemplary embodiments describe a system and method of marking the cardiac time intervals and display of the heart valve signals. Specifically, psychoacoustics are considered in identifying the separated cardiac vibration signals captured through the transducers. The system, the psychoacoustics, and a related method will be discussed in further detail below.

The exemplary embodiments provide a novel approach for small, portable, robust, fast and configurable source separation based software with transducer hardware. The use of a vibration signal pattern and novel psychoacoustics help bypass conventional issues faced by linear time invariant systems. Clinical indices of myocardial contractility can be categorized as follows based on pressure measurements (such as dP/dtmax), volume and dimension (such as stroke volume and ejection fraction) and systolic time intervals (such as pre-ejection period, left ventricular ejection time and isovolumic contraction time). dP/dtmax is the gold standard of measurement of myocardial contractility. Some of the cardiac time intervals can include Left Ventricular Systolic Time (LVST), Left Ventricular Diastolic Time (LVDT), Pre-atrial Diastolic Filling Time (PADT), Accelerated Atrial Filling Time (AAFT), QS1 (Electromechanical activation time), QS2, Pre-Ejection Period (PEP), Right Ventricular Systolic Time (RVST), Left Atrial Systolic Time (LAST), Right Atrial Systolic Time (RAST), Right Ventricular Ejection Fraction (RVEF), Right Ventricular Diastolic Time (RVDT), Left Atrial Diastolic Time (LADT), Right Atrial Diastolic Time (RADT), Systolic Time Interval (PEP/LVST), Mitral closing time, Mitral opening time, aortic opening time, or aortic closing time.

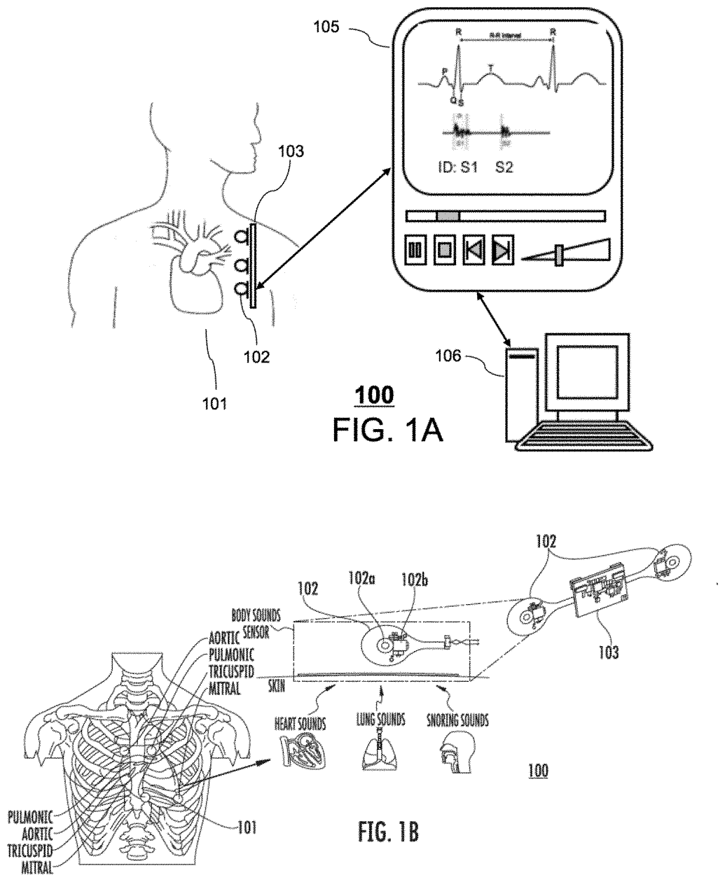

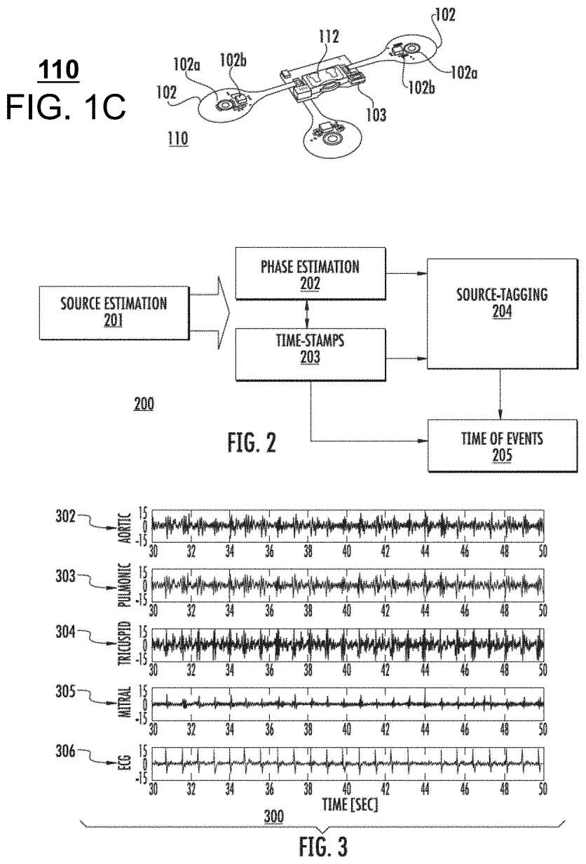

The exemplary embodiments of the system and method proposed here are shown in FIGS. 1A, 1B, and 1C. System 100 shown in FIGS. 1A and 1B is an embedded platform which can be any smart processing platform with digital signal processing capabilities, application processor, data storage, display, input modality like touch-screen or keypad, microphones, speaker, Bluetooth, and connection to the internet via WAN, Wi-Fi, Ethernet or USB. This embodies custom embedded hardware, smartphone, iPad-like and iPod-like devices. Area 101 in FIGS. 1A and 1B is the auditory scene at the chest locations. Array 102 in FIGS. 1A and 1B is the transducer array used to capture the heart signal(s). In some embodiments, the transducer array includes a pad that includes a vibration sensor such as a vibration sensor 102b and an electrode 102a for an ECG sensor. In some embodiments, the transducer array can include a single pad, two pads as shown in FIG. 1B or more than two pads as shown in FIG. 1C. In the particular embodiment of FIG. 1C, a transducer array 110 includes three pads (102) where each pad includes the vibration sensor 102b and the ECG electronic 102a. Other embodiments can include three or more pads where each pad would have at least a vibration sensor and optionally an electrode for the ECG sensor. Hardware 103 in FIGS. 1A-C is the wearable microprocessor hardware with digital signal processing capabilities, application processor, Analog to digital frontend, data storage, input modality like buttons, and wireless connection via Bluetooth, Bluetooth low energy, near field communication transceiver, Wi-Fi, Ethernet or USB.

Processor 112 shown in FIG. 1C comprises of the signal processing module on the wearable device that captures synchronized sensor data from the transducer array 102. The processor 112 is configured to save the synchronized sensor data to memory and communicate it with the system 100 for data transfer. Module 105 in FIG. 1A is the module that calculates vital sign from the input sensor stream coming from hardware 103 for the Heart rate, breathing rate, EKG signal, skin temperature, Blood oxygen level measurement, and associated vitals. The hardware 103 can optionally encrypt the raw sensor data for transmission to the cloud computing module 106. It can also communicate with a dashboard on module 105 or 106 for data exchange, login, alerts, notifications, display of processed data. Computing device 106 in FIG. 1A is the cloud module that processes the individual streams for eventual source separation. In some embodiments, the system 100 could include a connected display or other modality of display or presentation device. In some embodiments the system 100 allows a user to visually see the individual streams and information of the different cardiopulmonary signals.

The transducer array 102 can include multiple sensor transducers that capture the composite signal that includes the electrocardiogram signals, Blood oxygen signals, heart sounds, lung sounds and snoring sounds for example. The module 103 can be in the form of wearable hardware that synchronously collects the signals across the transducers and is responsible for the analog to digital conversion, storage and transmission to a portable unit 104. Note that the embodiments herein are not limited to processing the individual streams for source separation, identification and marking of the heart valve signals at the cloud computing module 106 only. Given sufficient processing power, the aforementioned processing can occur at the microprocessor hardware module 103, at the module 105, or at the cloud-computing module 106, or such processing can be distributed among such modules 103, 105, or 106.

The exemplary embodiments of the system and method proposed here for the source identification of the cardiopulmonary signals 200 are shown in FIG. 2. Block 201 indicates the separation of sources from the composite signals. Block 202 represents the phase estimation between the separated sources at each of the sensor position. Block 203 represents calculating the time stamps of individual sources at each heartbeat with respect to the synchronized EKG signal and the other sensor or sensors. Block 204 represents the source identification module responsible for tagging each of the separated source in individual heart beats to be one of the heart valve event, namely Mitral valve closing and opening, Tricuspid valve closing and opening, Aortic valve opening and closing, and the Pulmonic valve opening and closing. Block 205 represents the time marking module to estimate the time of occurrence of the above mentioned valve events with respect to the start of the EKG signal.

The exemplary embodiments of the system and method proposed here for the source identification of the cardiopulmonary signals from the composite signal 300 are shown in FIG. 3. Area(s) 101 in FIG. 1B indicate the locations at which the composite heart signal can be captured. A vibration signal 302 as charted on the first line in FIG. 3 represents a signal captured at the aortic auscultation location. A vibration signal 303 shows the vibration signal captured at the pulmonic auscultation location. A vibration signal 304 shows the vibration signal captured at the tricuspid auscultation location. A vibration signal 305 represents a vibration signal captured at the mitral auscultation location. The last or bottom line in FIG. 3 represents an electrocardiogram signal 306 captured. In some embodiments, note that the number of sensors used (such as in the sensor array 102 of FIG. 1), are less than the number of vibration sources. For example, 3 sensors can be used to ultimately extract signals for 4 (or more) vibration sources; or 2 sensors can be used to ultimately extract signals for 3 or 4 (or more) vibration sources; or 1 sensor can be used to ultimately extract signals for 2, or 3, or 4 (or more) vibration sources.

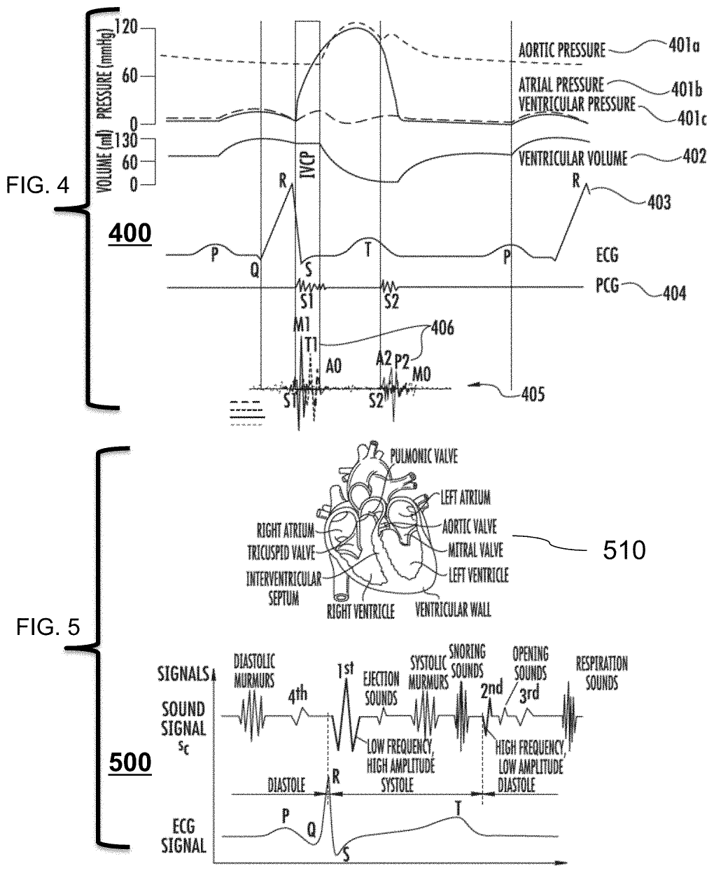

The exemplary embodiments of the system and method proposed here draw inspirations from biology with respect to the cardiac cycle in-relation with electrocardiogram and accelerometer transducer captured cardiac signal. In some embodiments, any cardiac waveform sensor can be used which is a more general term that includes accelerometer transducers or sensors. A timeline chart 400 in FIG. 4 shows a cardiac cycle. Lines or signals 401a, 401b, and 401c represent or indicate the pressure changes during a cardiac cycle for aortic pressure (401a), atrial pressure (401b) and ventricular pressure (401c) measured in measured in millimeters of mercury (mmHg). Line or signal 402 represents or indicates the volume changes during a cardiac cycle in milliliters (ml). Line or signal 403 represents or indicates the electrical changes during a cardiac cycle captured by an electrocardiogram. Line or signal 404 represents or indicates the acoustic changes during a cardiac cycle captured by an acoustic sensor such as a phonocardiogram or PCG. S1 represents the first heart sound or the “lub” sound and the S2 represents the second heart sound or “dub” sound. Line or signal 405 represents or indicates the vibration changes during a cardiac cycle captured by an accelerometer transducer (or cardiac waveform sensor) at the location of the device. Pattern 406 in FIG. 4 indicates the different valve opening and closing seen in line or signal 405 as captured by the accelerometer (or cardiac waveform) sensor or sensors. More specifically, a closer inspection of the pattern 406 reveals the closing of the mitral valve (M1) and tricuspid valve (T1) during the S1 or first heart sound and the closing of the aortic valve (A2) and pulmonary valve (P2).

FIG. 5 goes on to further show a representation 510 of the human heart relevant for the generation of the sounds and corresponding graph 500 representing the sounds belonging to coronary artery, murmurs, first sound, second sound, third sound, fourth sound, ejection sounds, opening sounds, respiratory sound, breathing, and snoring during individual heart beats, with respect to the electrocardiogram signal.

The exemplary embodiments of the system and method proposed here provide a source marking algorithm for the vibrations from the cardiohemic system. In some embodiments, the system next uses PCA to determine which source is associated with which event (e.g., Mitral closing & opening, Tricuspid closing & opening, Aortic opening & closing, Pulmonic opening and closing). The following describes the architecture for automatic source tagging and timing of valvular events. One way to identify which events are relevant to a source is by manually tagging the sources against the synchronized EKG signal and taking advantage of the timings relative to a QRS wave (identification of the S1 and S2 sounds using the EKG signal as the reference has been widely researched in studies). Another approach is an automatic tagging algorithm. The tagging is composed of a classifier preceded by a feature extraction algorithm. For the timing, the system exploits the computations of one of the feature extraction algorithms to obtain an energy contour from which the time location of a given event can be inferred. Because the embodiments here build upon having the ability to capture the signal at different locations simultaneously, to the proposed system exploits the relations among channels to extract additional information about the sources. Likewise, since some source separation algorithms where channels relations are associated with location, the system can leverage on the intrinsic relations among the channels to extract relevant information that helps the system distinguish among the events. In some embodiments, the system hypothesizes that phase information between channels is relevant for distinguishing among cardiac events since valves are located at different positions within the heart. Perhaps, one of the most distinctive features of the cardiac events is their relative order of occurrence, which repeats periodically with each heartbeat. Time information extracted from the set of sources can be utilized to localize the occurrence of each source signal within the heart cycle. Therefore, the features proposed here are conceived to provide three aspects: 1) Spectral information, 2) Relations among channels, and 3) Relations among events in the form of relative times of occurrence.

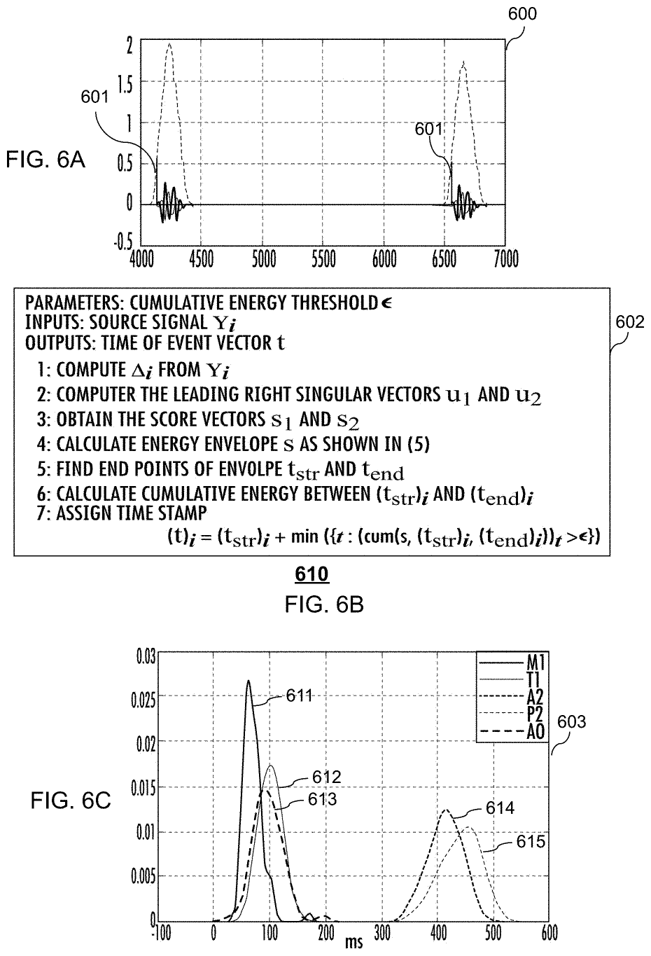

The automated timing is obtained from the separated sources. The embodiments can employ the eigenfilter approach described above to extract energy envelopes that can be easily detected and further processed to extract a time point. In this case, the system uses the two leading right singular vectors of the tap-delay matrix. It has been observed that, for a single channel, the first two right singular vectors of the tap-delay matrix contain oscillatory components with π/2 phase delay. This behavior can be extended to the two-channel case by noticing that the first half of the two leading singular vectors contain an oscillatory component of similar frequency with the above mentioned π/2 phase difference for channel 1, and that the same result applies to the second half for channel 2. From the above observation, we can consider the first 2 leading right singular vectors as a quadrature pair of eigenfilters. In other words, these filters have the same magnitude in frequency with a π/2 phase difference. The sum of instantaneous energies for the quadrature pair provides an energy envelope that, for the source signals, can be processed in a simple way to obtain time stamps on the occurrence of the events associated with the source. Let u1 and u2 be the two leading right singular vectors of Δi. Let s₁=Δᵢ u₁ and s₂=Δi u₂ be the score vectors. The energy envelope s can be calculated as (s)ₗ=(s₁)ₗ²+(s₂)ₗ². From the sparsity property of the heart sounds, it is possible to detect single heart beats from the energy contour s since the source signal is mostly zeroes followed by the oscillations related to the event at each heart beat. A simple marking procedure can be obtained by first detecting individual heartbeats and then processing the cumulative energy within a heartbeat to set a threshold that defines the marking point. Process 602 shown in the box 610 of FIG. 6B describes the procedure. A resulting time stamp (black vertical lines) 601 (in chart 600 of FIG. 6A) using the energy threshold can be marked. Notice that the endpoints of the Heart valve signal have been also detected as part of the procedure in determining the time stamps 601. The chart 600 shows the resulting markings using a cumulative energy to provide a threshold. In this case 1% of cumulative energy was selected to provide the threshold value. Chart 603 shows the time intervals found for the Mitral closing (611), Tricuspid closing (612), Aortic opening (613), Aortic closing (614) and Pulmonic closing (615).

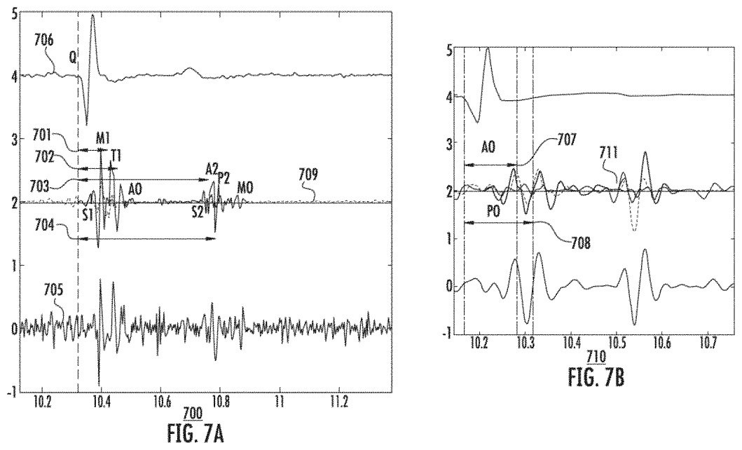

The exemplary embodiments of the system and method proposed here provide a source marking algorithm that allows from the explanation earlier for the marking of the Mitral valve closing (MC), Mitral valve opening (MO), Aortic valve opening (AO), Aortic valve closing (AC), Tricuspid valve closing (TC), Tricuspid valve opening (TO), Pulmonary valve closing (PC) and Pulmonary valve opening (PO) signals. The extracted individual valve vibration objects are aligned into a signal for each of the four valves across multiple heart beats. The chart 700 in FIG. 7A shows the source separation of heart valve opening and closing signals. Line 701 indicates the length or duration of the vibration signal for the Mitral valve closing (M1). Line 702 indicates the length or duration of the vibration signal for the Tricuspid valve closing (T1). Line 703 indicates the length or duration of the vibration signal for the Aortic valve closing (A2). Line 704 indicates length or duration of the vibration signal for the Pulmonic valve closing (P2). Signal 705 indicates the composite vibration signal captured by a particular transducer. Signal 706 indicates the EKG signal captured by the system. Referring to chart 710 of FIG. 7B, the Line 707 indicates the length or duration of the vibration of the Aortic valve opening (AO). Line 708 indicates the length or duration of the vibration of the Pulmonic valve opening (PO). Further note that the lines or signals 709 in FIG. 7A or 711 in FIG. 7B are actually several separated superimposed signals representing the vibration signals from separate sources coming from the mitral valve, tricuspid valve, aortic valve, and pulmonary valve (using less than 4 vibration sensors to extract such separated signals in some embodiments.

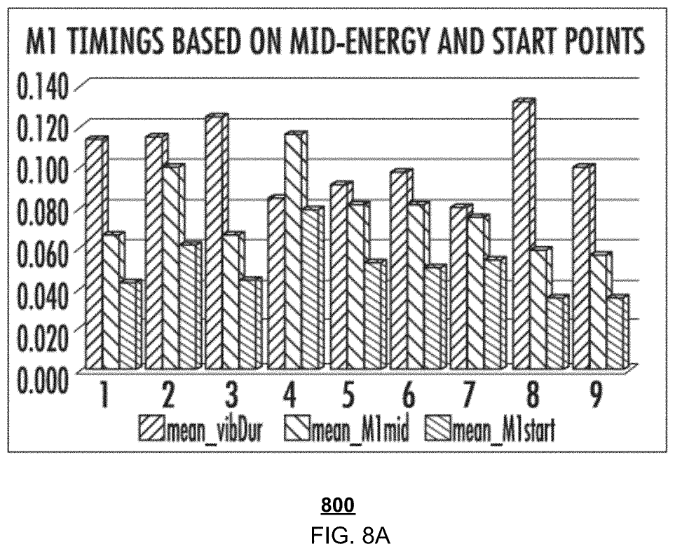

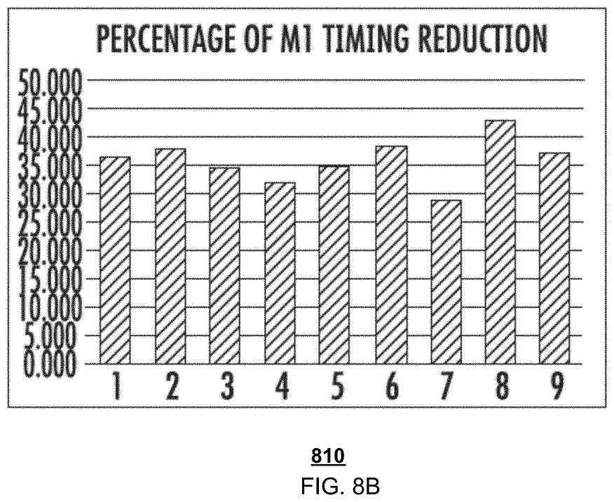

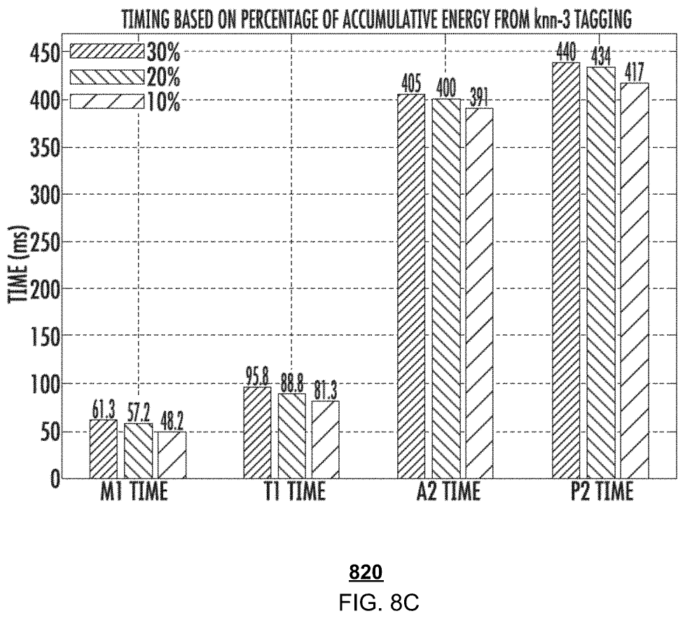

It was observed that peak of T1 timing distribution is close to that of AO. The reason is that the length of M1 and T1 Source Separation vibrations is longer than the length of AO Source Separation vibrations. So when the mid-point of accumulative energy is calculated, M1 and T1 timings are already shifted forward and don't represent the start of the vibration. Such a timing shift exists for AO but it's not as big as M1 and T1. To verify and compare, the following time information on some patients helps provide different approaches: Mean length of M1, T1 vibration, Mean start point of M1, T1 vibration, Mid-energy point is obtained from PCA algorithm. A shift back in timing of M1, T1, A2, P2 by reducing the 50% of accumulative energy to 30%, 20%, and 10%. The results are demonstrated in FIGS. 8A, 8B and 8C.

In the exemplary embodiments, a novel way of calculating the timing of the source separated individual heart vibration events from the composite vibration objects captured via multiple transducers is used to work on a single package, easy-to-use and portable device.

The exemplary embodiments develop a novel method of source timing, which in one embodiment using the Pulmonary and Aortic, and in addition possibly the Tricuspid and Mitral auscultation locations, lends the system capable of calculating the time intervals of individual valve events from the vibrations with respect to the electrocardiogram.

The exemplary embodiments develop a novel method of time interval calculation, which in one embodiment using the Pulmonary and Aortic, and in addition possibly the Tricuspid and Mitral auscultation locations, lends the system capable of marking the time of occurrence of the individual valve events with respect to the electrocardiogram. The novel method lends the system capable of measuring the cardiac time intervals.

The exemplary embodiments develop a novel method of providing time intervals of individual valve signals over time. The novel method allows for both short-term and long-term discrimination between signals. Short-term pertains to tracking individual stream when they are captured simultaneously as part of the composite signal. Long-term tracking pertains to tracking individual streams across multiple heart beats, tracking valve signals as they transition in and out during each cardiac cycle.

The exemplary embodiment of system and method described is the development on an embedded hardware system, the main elements required to capture body sounds are the sensor unit that captures the body sounds, digitization, and digital processing of the body sounds for noise reduction, filtering and amplification. Of course, more complicated embodiments using the techniques described herein can use visual sensors, endoscopy cameras, ultrasound sensors, MRI, CT, PET, EEG and other scanning methods alone or in combination such that the monitoring techniques enable improvement in terms of source separation or identification, and/or marking of events such as heart valve openings, brain spikes, contractions, or even peristaltic movements or vibrations. Although the focus of the embodiments herein are for non-invasive applications, the techniques are not limited to such non-invasive monitoring. The techniques ultimately enable diagnosticians to better identify or associate or correlate detected vibrations or signaling with specific biological events (such as heart valve openings and closings, brain spikes, fetal signals, or pre-natal contractions.)

The exemplary embodiments herein provide a method and system based on a technique to identify the separated cardiopulmonary signals, to extract information contained in vibration objects. In one embodiment, known under machine learning, auditory scene analysis, or spare coding approaches to the source separation problem. Data is obtained using a tri-axial accelerometer or multiple tri-axial accelerometers placed on different points of torso.

Examples of cardiac vibration objects are the first sound, the second sound, the third sound, the fourth sound, ejection sounds, opening sounds, murmurs, heart wall motions, coronary artery sounds, and valve sounds of the Mitral valve opening and closing, Aortic valve opening and closing, Pulmonary valve opening and closing, Tricuspid valve opening and closing. Examples of the pulmonary vibration objects are the respiratory lung sounds, breathing sounds, tracheobronchial sounds, vesicular sounds, Broncho vesicular sounds, snoring sounds. A portion of the energy produced by these vibrations lies in the infra-sound range, which falls in the inaudible and low sensitivity human hearing range. A portion of the energy produced by these vibrations falls in the audible hearing range. Accelerometer transducers placed on the chest capture these vibrations from both these ranges.

Source identification analysis in accordance with the methods described herein identify individual vibration objects described above from the source separated vibration signals. The individual vibration signals are identified to be from the mitral valve, aortic valve, tricuspid valve, the pulmonary valve, coronary artery, murmurs, third sound, fourth sound, respiratory sound, breathing, and snoring during individual heart beats. The identified signals are marked to indicate their start with respect to the start of the EKG.

The embodiments can include different source identification techniques specifically used for tagging the individual cardiopulmonary signals for application in a non-linear time variant system, such as Principal component analysis, Gabor filtering, Generalized Cross Correlation (GCC), Phase transform (PHAT), ROTH, SCOT and Band Filtering. Using 1) Spectral information, 2) Relations among channels, and 3) Relations among events in the form of relative times of occurrence.

The exemplary embodiments provide a novel approach for small, portable, robust, fast and configurable source separation based software with transducer hardware 103, 203. The use of the vibration signal pattern and novel psychoacoustics help bypass conventional issues faced by linear time invariant systems.

The following are the various types of heart disease that can be diagnosed and treated using the identifies signals, namely, Coronary artery disease, Heart murmurs and valve abnormalities, Heart failure, Heart rhythm abnormalities (arrhythmias), Vascular disease, congenital heart disease, and Risk factor modification. A physician can work with patients to perform a comprehensive evaluation and design a personalized plan of care aimed at keeping them healthy.

In another embodiment, the source can be identified by manually tagging them against the synchronized EKG signal and taking advantage of the timings relative to the QRS wave. This way, however is usually slow and time consuming and an automatic tagging algorithm is thus preferable. Since the different heart sounds comes from different locations in the heart, it is expected that each source will have a unique phase relation between the sensors that are located at fixed points during the data gathering phase. This phase relation can be made evident by using signal representation with a dictionary suited for highlighting frequency and phase relations with a greedy algorithm such as the matching pursuit. Our algorithm performs a Gabor analysis in the source separated signals using a finite Gabor Dictionary of fixed frequencies and with variable phase delay. The system finds the delay that minimizes the reconstruction error for each source and creates a group of features that will be incorporated into a decision-making and classification algorithm. The classification algorithm will combine the features extracted by the Gabor analysis with other features that comes from the PCA and cross correlation analysis that uses a set of manually tagged patient's tracks as training. The system is composed of three modules or stages: A suitable Gabor dictionary is created that can serve as the basis representation of the signals. An optimization algorithm aims to find the delay that will minimize the reconstruction error in a giving delay range. Collection and organization of all the features extracted from the second stage. For each one of the stages different techniques were tried in order to achieve the best results. The Gabor dictionary selected has the function:

$$G ( θ , n ) = {e}^{- \frac{1 {( n - θ )}^{2}}{2 {σ}^{2}}} cos ( 4 π \frac{n - θ}{f} )$$

In the equation θ is the delay, σ is the Gaussian decay of the atom and f is the frequency of operation of the Gabor. The system initially searched for the Gabor atoms that best represented the signal by doing a sweep in frequency and selecting those Gabor atoms that produced a larger weight matrix after the matching pursuit. The system was subsequently changed to a group of fixed frequency Gabor atoms exploiting the fact that for the M1, T1, A2 and P2 sounds vibration reside mainly in the range of 50-300 Hz and the Aortic opening vibration resides mainly in the low frequency range (from 0-50 Hz). The fixed Gabor approach sacrifices a little bit of reconstruction error in favor of a faster computation and easier analysis since the frequencies were fixed. The first optimization technique used was the, Gradient Descent algorithm: A first-order optimization algorithm that looks for the minimum of a given function by taking steps proportional to the negative of the gradient. The system is very dependent on the initial guess and number of minima of the system. A second approach was using the minimize function, which uses the Polack-Ribiere algorithm of conjugate gradients to compute search directions, and a line search using quadratic and cubic polynomial approximations. The system is also dependent on the initial value of the search but is more efficient in choosing the parameters to find the minimum. Finally, a brute force approach was implemented that sweeps the signal with different delays and then selects the one that resulted in a minimum function. In order to improve speed on this algorithm, a broad search is done first and then a refined search is done around the minimum point found by the broad search.

The exemplary embodiments of the system and method proposed here provide a source identification algorithm for the vibrations from the cardiopulmonary system. In order to find the time stamps for events such as Mitral closing & opening, Tricuspid closing & opening, Aortic opening & closing, Pulmonic opening and closing, we look at all the individual source separated signals 701 to 706 of the composite signal 707 and first tried to find the location of max peak in the SS signal for each source and then find delay between two channels. 708 shows the frequency spectrum of the source separated signals. Cross correlated vibrations in aortic and pulmonic channels for each interval for each source are calculated to find a consistent delay between two channels 709. Given the start of QRS and end point of each vibration, the vibration(s) within this interval is cross correlated with all vibrations in each source. This is done for both aortic and pulmonic channels. At the end, Principle Component Analysis (PCA) was applied to find the timing information and delay between two channels. PCA uses SS signal from each source in each channel to find the template which represents majority of the vibrations within that source. The template is then cross correlated with the whole source and max of PCA signal in each interval is found and compared with the start of QRS. In another implementation, In the second attempt, SS signals from two channels but the same source are fed into PCA to find the template. Then aortic template is used for both channels' cross correlation to identify the different vibrations into valve events, breathing sounds, and vibrations of the heart walls. To accurately estimate M1, T1, A2, P2, A0, P0 from the frequency signal captured by digital accelerometer on the wearable, several sub-frequencies are considered. Some are noted here for example: 0-30 Hz, 0-60 Hz, 30-150 hz, 30-250 Hz.

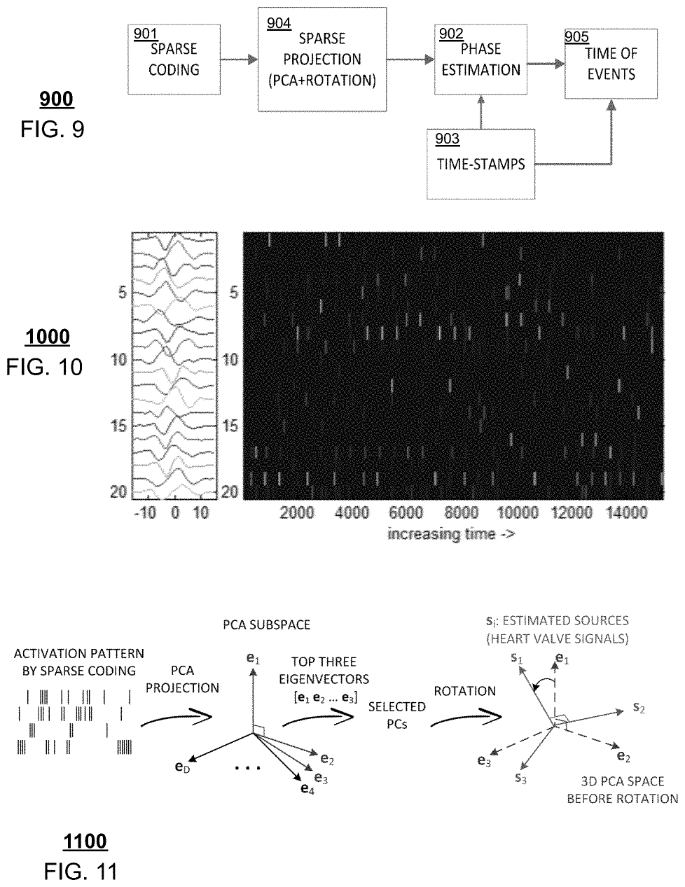

The exemplary embodiments of the system and method proposed here provide a source marking algorithm for the vibrations from the cardiopulmonary system. Next step is to use PCA to determine which source is associated with which event (Mitral closing & opening, Tricuspid closing & opening, Aortic opening & closing, Pulmonic opening and closing). We describe the architecture for automatic source tagging and timing of valvular events. One way to identify which events are relevant to a source is by manually tagging the sources against the synchronized EKG signal and taking advantage of the timings relative to the QRS wave (identification of the S1 and S2 sounds using the EKG signal as the reference hast been widely researched in studies. Another approach is an automatic tagging algorithm. The tagging is composed of a classifier preceded by a feature extraction algorithm. For the timing, we exploit the computations of one of the feature extraction algorithms to obtain an energy contour from which the time location of a given event can be inferred. Because our work builds upon having the ability to capture the signal at different locations simultaneously, we want to exploit the relations among channels to extract additional information about the sources. Likewise some source separation algorithms where channels relations are associated with location, we leverage on the intrinsic relations among the channels to extract relevant information that helps us distinguish among the events. We hypothesize that phase information between channels is relevant for distinguishing among cardiac events since valves are located at different position within the heart. Perhaps, one of the most distinctive features of the cardiac events is their relative order of occurrence, which repeats periodically with each heartbeat. Time information extracted from the set of sources can be utilized to localize the occurrence of each source signal within the heart cycle. Therefore, the features we propose here are conceived to provide three aspects: 1) Spectral information, 2) Relations among channels, and 3) Relations among events in the form of relative times of occurrence. We describe a feature extraction algorithm based on multichannel Gabor basis decomposition using matching pursuit, and a second algorithm that uses eigen-decomposition of a covariance matrix extracted from the source signals to obtain a cross correlation function between channels. For the gabor basis, consider the reconstruction of a 2-channel signal x (t). In this approach we want to extract a phase relation between the sensors which are located at standard aortic and pulmonic auscultation positions. As we mentioned above, we are interested on features that reflect spectral content as well as the channel interrelations. In the two channel case, each basis function is a pair of Gabor functions with equal frequency f and envelope width h and a phase difference θ. The tunable parameter θ can be swept across a range of values for which the matching pursuit reconstruction error can be obtained. The behavior of the reconstruction error for a source signal over different values of θ provides information about the phase different between the two channels for the particular source signal. In particular, the value of θ that attains the minimum error within the defined range of values is taken as the optimal channel delay, and the time average of the activation coefficients obtained by matching pursuit at the optimal 8 provide and spectral characterization of the source. For the feature Extraction by Self-Similarity Template Based-on PCA, we define a self-similarity using the concept of eigenfilter. Let yi the a two-channel source signal defined in (1), and let Yi its discretized version of size N×2. For filter of length Nw, we compute the tap-delay matrices Δ(j). The joint-channel eigenfilter correspond to the leading right-singular vector u of the tap-delay centered matrix Δi obtained by removing the mean of each column of Δi. The eigenfilter u can be split into the first and second channel components. The first channel component u(1) corresponds to the first Nw entries of u, and u(2) to the remaining entries going from index Nw+1 to last index 2Nw. The proposed feature is the cross-correlation function between u(1) and u(2). Note that this function contains the main frequency components of the source signals expressed in the time domain and the peak provides information about the channel relations. For different delays between the two channels the cross correlation peaks at different locations, accordingly. To extract relative time information contains cues for the classification of the cardiac events, we adopt a simple approach that uses the energy contours. The process consists of three basic steps: Compute energy contours for all source signals, Compute timings for each source signal, Compute features of source i by averaging the sum of the remainder sources centred at the timings. The energy of each source signal Yi is calculated using the leading, quadrature pair of right singular vectors of their corresponding tap-delay matrix. For classification, a set of training exemplars (sources) have been manually tagged with the respective events. Each source signal is then represented by the feature vectors described above. Test have been carried out on pooled covariance linear discriminant analysis, quadratic discriminant analysis, k-nearest neighbours and support vector machines both linear kernel and Gaussian kernel. The features described in the previous sections can be used in different manners for tagging, for example: Tagging using only Gabor-based features, Tagging using only self-similarity features, Tagging using only time-based features, Tagging using all Gabor-based, self-similarity, and time-based features. There are even sub cases of the situations considered above, For instance, the combination of Gabor and self-similarity features can be done on a single classifier or using ensembles. The first validation examples only address the cases in the bullet items. In addition to the classifier, preprocessing of the feature vectors is also performed. A centering vector X_{cent} is calculated using the mean over the training set Xₜᵣₐᵢₙ. This step is followed by a linear transformation based on PCA to produce a much smaller dimension feature vector. The preprocessing is summarized in Algorithm 1, FIG. 9. The choice of p is typically driven by the training data itself. The singular values of Xₜᵣₐᵢₙ can be used to decide the dimensionality of the transformed feature vectors after.

The exemplary embodiments of the system and method proposed here provide a source marking algorithm that allows from the explanation earlier for the marking of the Mitral valve closing (MC), Mitral valve opening (MO), Aortic valve opening (AO), Aortic valve closing (AC), Tricuspid valve closing (TC), Tricuspid valve opening (TO), Pulmonary valve closing (PC) and Pulmonary valve opening (PO) signals. The extracted individual valve vibration objects are aligned into a signal for each of the four valves across multiple heart beats. 800 in FIG. 8 show the source separation of heart valve opening and closing signals. 801 indicate the vibration signal for the Mitral valve closing. 802 indicate the vibration signal for the Tricuspid valve closing. 803 indicate the vibration signal for the Aortic valve closing. 804 indicate the vibration signal for the Pulmonic valve closing. 805 indicate the composite vibration signal captured by a particular transducer. 806 indicate the EKG signal captured by the system. 807 indicate the vibration of the Aortic valve opening. 808 indicate the vibration of the Pulmonic valve opening.

The exemplary embodiments of the system and method proposed here provide a source marking algorithm for the vibrations from the cardiopulmonary system, using information about the time of occurrence of the event. Automated finding of M1, T1, A2 & P2: After going through the timing plots of different patients, it was decided to first determine A2 and P2 timing and their corresponding sources and then find M1 and T1 from the rest of sources. Automatic A2 & P2 finding: 1) The number of zero-crossings is found for each SS source. Then the noisy source that is associated with the maximum zero-crossing is discarded. 2) Automated calculation of QRS onset points for all heartbeats in each source. 3) Automated calculation of beginning and ending points of all vibrations in each source. 4) Applying PCA approach on all sources that outputs the time difference between QRS onset and peak of PCA signal (timing vector) as well as delay between aortic and pulmonic channels. 5) Based on probability density estimation of each source's timing vector, strong peak(s) that correspond(s) to timing(s) more than 300 ms are accepted as A2 and P2 candidates and those sources that don't satisfy this condition are discarded. Also new candidate timings are sorted in ascending order. 6) Variation, length of samples more than 300 ms (L) and some other measurements are calculated from accepted timing vectors of accepted sources and are stored in a structure for further analysis. 7) If there is only one strong peak in probability density estimation plot whose timing is more than 300 ms, then a variable called “ind_both” is set to 1. If there are two or more strong peaks whose timing is more than 300 ms, if max peak's timing is over 300 ms, then “ind_both” variable is set to 1 but if max peak's timing is lower than 300 ms but another strong peak's timing is more than 300 ms, then “ind_both” is set to 0. If there are two or more sources whose “both_indic” variables are equal to 1, then “both_indic”=1. 8) In case of having no valid timing, A2 and P2 are set to zero. 9) In case of having one valid timing, if L>6 then it is marked as A2 and its corresponding source number is also saved. P2 is set to zero. 10) In case of having two valid timings:

```
      if both_indic =1 ,
        if L1 >= 6 samples, then A2 is set to first timing in sorted timing vector Else
A2=0
        if A2=0 and L2 >=6, then A2 is set to second timing in sorted timing vector Else
P2=0
        if A2 ~= 0 and L2 >=6, then P2 is set to second timing in sorted timing vector
Else P2=0
if both_indic ~=1 ,
  if L1 >= 4 samples & V1 <=800 then A2 is set to first timing in sorted timing vector Else
A2=0
  if A2=0 and L2 >=4 & V1<=800 , then A2 is set to second timing in sorted timing
vector Else P2=0
  if A2 ~= 0 and L2 >=6 & V2<=800 then P2 is set to second timing in sorted timing
vector Else P2=0
    11)In case of having more than two valid timings,
if both_indic =1
for loop: if L1 >=6 & V1<=800, then A2 is set to current timing in sorted timing vector;
break Else A2=0; End of for loop
    if A2=0, then P2 = 0;
    if A2~=0 & last timing in the sorted timing vector
          if L >=6 & V1<=800, then P2 is set to last timing in the sorted timing vector
    if A1~=0 & there are two timings after A1 timing,
          if V2<V1, then P2 is set to second timing after A2.
          Else P2 is set to first timing after A2.
    if A1~=0 & there are more than two timings after A1 timing,
          if L1>=6 & V1<=800 , then P2 is set.
if both_indic ~=1 ,
for loop: if L1 >= 8 samples & V1<=1000 then A2 is set to current timing in sorted
timing vector Else A2=0; End of for loop.
  if A2=0, then P2 = 0;
    if A2~=0
for loop: if L >=8 & V1<=1000,
          then P2 is set to current timing in the sorted timing vector; break
          Else P = 0;
End of for loop
```

At the end A2 and P2 timings and their corresponding source numbers are saved in an excel with a long with patient name.

### Automatic M1 & T1 Finding:

1) The same previous steps from 1 to 7 are implemented in this phase
8) In case of having no valid timing, M1 and T1 are set to zero.
9) In case of having one valid timing, if L>6 and timing is less than 120 ms, then it is marked as M1 and its corresponding source number is also saved. P2 is set to zero.
10) In case of having two valid timings,

```
if both_indic =1 ,
  if L1 >=6, then M1 is set to first timing in the sorted timing vector Else M1 =0
  if M1 =0 & L2 >=6, then M1 is set to second timing in the sorted timing vector and
T1=0
  if M1~=0 & L2>=6 and second timing is less than double the first timing
          then T1 = second timing in the sorted timing vector Else T1 =0
if both_indic ~=1
L1>=4 & V1<=800, then M1 is set to first timing in the sorted timing vector Else M1 =0
If M1=0 & L2>=4 & V2<=800, then M1 = second timing in the sorted timing vector Else
T1 =0
If M1~=0 & L2>=4 & V2<=800, then T1 = second timing in the sorted timing vector Else
T1 =0
    11)In case of having more than two valid timings,
if both_indic ~=1 ,
for loop: if L >=8 & V<=1000, then M1 is set to current timing in the sorted timing vector
          Else M1 =0; break; end of for loop
  if M1 =0, then T1 = 0;
for loop: if M1~=0 & L >=6 & V<=1000, then T1 is set to current timing in the sorted
timing vector; break, end of for loop
if M1~=0 & L2>=6 and second timing is less than double the first timing
          then T1 = second timing in the sorted timing vector Else T1 =0
if both_indic =1
low_ var = Find sources with variations less than 15.
From these low_var sources, find those whose length are more than 9
samples(len_low_var).
If length of low_var >=2 & len_low_var >=2
          M1 is set to the timing corresponding to the lowest variation
          T1 is set to the timing corresponding to the second lowest variation
Else
for loop: If L >=5 & V<800, then M1 is set to the current timing in the sorted timing
vector Else M1=0
If M1 =0, then T1 =0
If M1~=0
for loop:
If there is only one timing after M1 & L >=5 & V <=800, then T1 = current timing in the
sorted timing vector; break
If there are two timings after M1,
if V1 < 300, then T1 = first timing after M1 in the sorted timing vector; break
Elseif V2<V1, then T1 = second timing after M1 in the sorted timing vector; break
Else, T1 = first timing after M1 in the sorted timing vector; break
If there are more than two timings after M1,
if V1 < 5, then T1 = current timing after M1 in the sorted timing vector; break
Elseif V2<V1, then T1 = current timing after M1 in the sorted timing vector; break
Else, T1 = current timing after M1 in the sorted timing vector; break
---
```

Automated finding of Aortic opening (AO):
Track 1 with 2 channels 8 by 2 by N
M1, T1 timings and EKG times
Remove the noisy source using max zero-crossing. Data down to 7 by 2 by N
Sub-source markings including start and end of vibrations, envelope and max peak
Save to non-zero-source vector for both channels
Remove missing start and end points and too large ones by comparing between two channels as well as considering each source
Calculate PCA for two channels and all vibrations. Outputs are delay_vec, PCA signal and delay between two channels

### Begin AO Detection

Select above data for AO channel
Remove zero delay values
Remove delays between two channels that are greater than 50 ms and count <4
Remove positive delays between two channels and count <5
Calculate ksdensity
Find max peak as well as other peaks that their distance together is more than 5 samples
Keep negative delays if delay is less than 150 ms
Calculate ksdensity of event_vec
Find more max peak and peaks>half of max peak
Find peaks>10 ms and with 0.05 of max probability value
Save all the candidate sources with negative delay btw two channels
Select peaks that fall in range of M1 and M1+95 ms (if M1=T1=0, in range 20 to 150 ms)
Sort all timings and save
Find AO
   First case: If there is only on candidate source:
1) If delay variation<500 but source variation>1000,
2) remove timings>200 ms,
3) If length of samples>4, remove samples whose timings relative to median are >60 ms
4) If empty timing vector:
I. then get the original timing vector, calculate kdensity and find max and other peaks whose probability is more than half of max's probability
II. If time diff btw AO candidate and max peak's timing <10 ms,

set max's peak timing as ref time, remove samples with diff more than 50 ms relative to ref time and update the AO candidate and its variation.
III. If time diff btw AO candidate and max peak's timing >10 ms,

find a qualified peak and its corresponding timings that is closest to AO candidate, set that peak's timing as ref time, remove samples with diff more than 50 ms relative to ref time and update the AO candidate and its variation.
5) If non-empty timing vector:
I. calculate kdensity and find max and other peaks whose probability is more than half of max's probability. Find the one that is closest to candidate, set is as ref time, remove samples with diff more than 50 ms relative to ref time and update the AO candidate and its variation.
6) Check if var_delay<500 && var_SRC<900 && AO_candidate<=M1+96
   Second case: If there are two candidate sources:
   The following steps from the first case are implemented:
1, 2, 3 with 50 ms threshold, and 6
After founding all valid AO timings from all candidate sources, the closet AO candidate to “M1+30 ms” is selected.
   Third case: If there are more than two candidate sources:
   The following steps from the first case are implemented:
1, 2, 3 with 90 ms threshold, 4 with 90 ms threshold, and 6
After founding all valid AO timings from all candidate sources, the closet AO candidate to “M1+30 ms” is selected.

To improve the results in the exemplary embodiment we develop AO detection using new PCA approach such as: Mean removal, Min-Max removal, Different window length for correlation. Detailed analysis on delay calculation from cross-correlation template. Source tagging using PCA features and different classifiers on both tagged and untagged tracks for both High Pass and Low Pass. Defined two timing calculation algorithm 1) with threshold 2) without threshold and got the timing distributions for different test cases. To improve AO detection using new PCA algorithm, a few different tests were done such as: Removed the mean of each source's template (X matrix) before calculating covariance matrix. For some sources, cross-correlation template shows zero delay at a discontinued point although there is a delay between peaks of templates. Removed min-max of eigenvector before performing cross-correlation showing a continuous behavior with a delay. Then ran different SSs on the same patient to see if these observations are consistent. There are different scenarios when calculating the delay such as: Maximum peak is positive or negative, The Delay of Max peak is positive or negative. Our approach is to select positive maximum peak with the positive delay. The goal is to tag or label the sources with an event (HP: Ml, T1, A2, P2, LP: AO). The cross-correlation signals generated by new PCA algorithm are used as features to train the classifiers and then tag sources of a test signal. Similar approach can be extended for the identification of other cardiopulmonary signals, like coronary artery sounds, snoring, murmurs, respiration sounds in one embodiment.

Referring to FIGS. 9-11, The exemplary embodiments of the system and method proposed here provide a source marking algorithm for vibrations from the cardiohemic system. In some embodiments, the system includes a sparse projection of a deficient activation pattern given by an underdetermined source separation algorithm (see FIG. 9). The activation patterns (see FIG. 10) are projected to a possibly lower dimensional space of reconstructed sources. The projection consists of an unsupervised principal component analysis (PCA) metric projection, followed by a regularization step to make the projection loadings sparser. The regularization is performed by an orthogonal rotation of PCA subspace, and helps to better understand the rotated space which is now aligned with the space of valvular events (see FIG. 11).

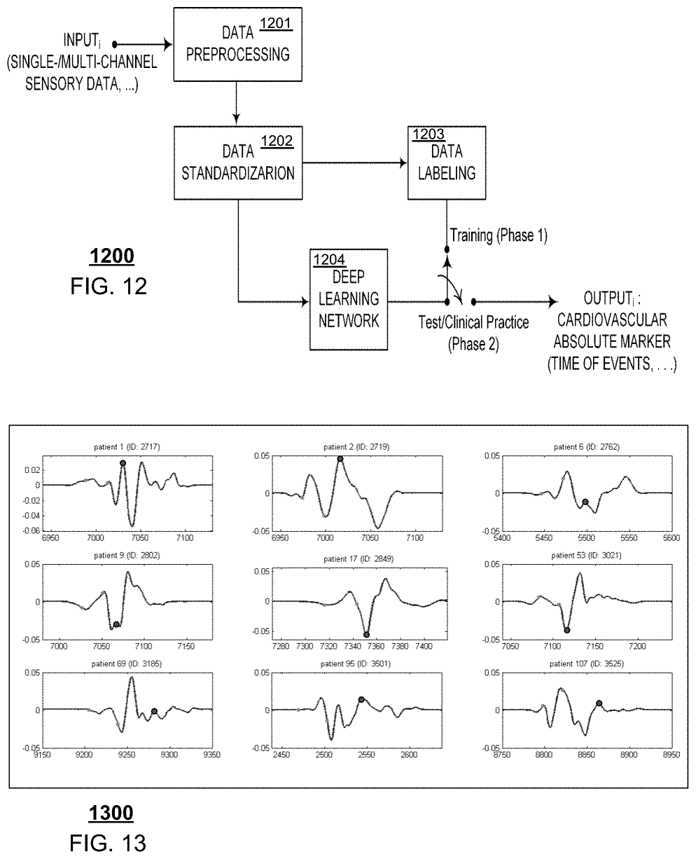

Referring now to FIGS. 12 and 13, diagnosis of cardiovascular diseases, including heart failure, are currently based on medical experts' interpretation of patient's physical examination and medical records using a well-developed flow-chart, comparing patient's conditions with a typical taxonomy of known conditions. However, this diagnostic procedure is not always accessible, and may be expensive or inefficient—as it is error-prone. An alternative approach to access appropriate medical diagnostic services is to use artificial intelligence and design rule-based expert systems or graphical models to consistently follow the same diagnostic procedure performed by a physician. However, designing a system to imitate the reasoning processes of human experts could be very challenging, as it requires significant rule extraction and feature engineering.

An alternative to artificial intelligence is deep learning that has recently produced considerable breakthroughs proven to be very efficient for image and signal analysis, like image classification and segmentation, speech recognition, language translation, and structured data analysis. Deep learning is based on deep neural networks with hierarchical intermediate layers of artificial neurons, which can progressively extract increasingly complex features. By learning very complicated input-output relationships, deep learning can outperform systems that are based on manual feature extraction, like rule-based expert systems.

A prerequisite for using deep learning is that there must be a thousand to millions of well-labeled data points (pairs of input-output) to train the deep network. With the new advances in cardiovascular technologies, which are able to capture large quantities of patients' data, this prerequisite is met. A recent survey (by Bizopoulos, P. and Koutsouris, D., 2018. Deep Learning in Cardiology. IEEE reviews in biomedical engineering, 12, pp. 168-193) summarizes the applications of deep learning in cardiology, where it is applied to patients' structured data, and signal and image modalities in cardiology of heart and vessel structures.

Despite of superior performance of deep learning for some cardiovascular applications, their use as a diagnostic black box prevents them to integrate in the clinical practices (as reported by Miotto, R., Wang, F., Wang, S., Jiang, X. and Dudley, J. T., 2017. Deep learning for healthcare: review, opportunities and challenges. Briefings in bioinformatics, 19(6), pp. 1236-1246).

We introduce a system (see FIG. 12) based on deep learning (DL) networks, which is applicable to any sensory database, image, motion image/video, single or multichannel sensor signals (from sensors such as cardiac waveform sensors, ECG, accelerometers, gyroscopes, acoustic microphones, micro-electro-mechanical systems-MEMS, microwave, radar, radio-frequency, doppler, or Near Field Communication-NFC) and structured data and after proper training with pairs of cardiovascular input-output, can produce absolute markers, like heart valvular events (e.g., Mitral closing & opening, Tricuspid closing & opening, Aortic closing & opening, Pulmonic closing & opening), E/e′, ejection fraction (EF), global longitudinal strain (GLS), left atrial volume index, different cardiac time intervals (CTI's), mean Pulmonary Artery pressures (mPAP), systolic Pulmonary Artery pressures (sPAP), and diastolic Pulmonary Artery pressures (dPAP), Pulmonary Wedge Capillary Pressure (PCWP), Cardiac Output and stroke volume. This is a totally different perspective of using deep learning, because we train the system with absolute markers so the immediate outputs of the deep learning after training are absolute markers. This is consistent with the concept of evidence-based medicine and partially satisfies the ethical and legal issues of using machine learning and artificial intelligence methods in clinical practices, which is currently a big challenge for their practical application (as discussed by Lee, E. J., Kim, Y. H., Kim, N. and Kang, D. W., 2017. Deep into the brain: artificial intelligence in stroke imaging. Journal of stroke, 19(3), p. 277). Note that E/e′ is the ratio between early mitral inflow velocity and mitral annular early diastolic velocity on echocardiography and is the best known non-invasive estimation of LV (left ventricle) filling pressures.

The embodiment of the system and method proposed here includes a preprocessing step (noise removal, etc.), a data standardization that brings the preprocessed data to a common format (normalization, scaling, windowing, etc.). It also includes a manual or automatic data labeling step to extract the desired outputs (labels) for each particular input (FIG. 2, shows one such embodiment of the inputs, namely, the Aortic valve openings). On training session (Phase 1) the deep learning module learns from pairs of inputᵢ/outputᵢ. At the end of training session the system is ready for test and clinical application (Phase 2).

The embodiment of the system and method proposed here is applicable for longitudinal monitoring of absolute markers over time, when the times series of markers provided over hours, days or weeks indicates if the patient's symptoms is improving/worsening.

The embodiment of the system and method proposed here can be used in a multidisciplinary framework to generate combined outpatient markers, like acute decompensated heart failure, heart failure with preserved ejection fraction (HFpEF) for diastolic dysfunction, heart failure with reduced ejection fraction (HFrEF) for systolic dysfunction pulmonary hypertension, different grades of diastolic dysfunction I, II, III, different grades of Mitral Valve Regurgitation (mild, moderate, moderately severe, or severe), different grades of Mitral Valve Stenosis (mild, moderate, or severe), different grades of Aortic Regurgitation (mild, moderate, moderately severe, or severe), different grades of Aortic Stenosis (mild, moderate, or severe), congestion in the lungs, Coronary Artery Disease, Lung Disease, Ischemic Cardiomyopathy, Left Ventricular Hypertrophy, Sleep Apnea, Angina, and Myocardial Infraction.

The deep learning core of the embodiment of the system and method proposed here, can be deep feed forward (DFF), deep belief network (DBN), deep convolutional network (DCN), deep convolutional inverse graphics network (DCIGN), Deep Boltzmann Machine (DBM), or any other deep structure.

Although the form factor of having a non-invasive device with multiple sensor pads as shown in FIGS. 1B and 1C are effective, embodiments using a single pad or patch can provide greater ease of use and reliability in light of the increasing healthcare demands.

Similarly, other cardiac sensor modalities capture signals correlated with the cardiohemic events, including but not limited to electrocardiogram (ECG), impedance cardiography (ICG), phonocardiography (PCG), photoplethysmography (PPG), seismocardiography (SCG), ballistocardiography (BCG), or gyrocardiography (GCG).

Hospitals are the community's anchor and provide a safe haven in times of emergency. And the healthcare system is seeing an unprecedented number of patients revolving through its doors. 13 million hospital visits (8 million Hospital Outpatient, 4 million Emergency Department, and 1 million hospitalizations) and an incredible amount of re-hospitalizations. In spite of progress in effective medical therapy, 25% of all Medicare population patients are readmitted within 30 days of hospitalization. The majority of these admissions are due to congestive exacerbations due to fluid overload in heart failure patients. The cost of proving all this care is astronomical. The indirect cost due to lost wages or loss of work is almost equal at 300 billion, and estimated to grow to $800 billion by 2030. This problem is not isolated but spread across all the 50 United States in different intensity. Adding to this, economic implications favoring early hospital discharge have led to an ever-increasing demand to send patients home for recuperation. Patients are now routinely released on their fourth day, thereby making post monitoring from home ever more essential. Patients discharged following congestive heart failure, myocardial infarction, coronary artery bypass grafting surgery or other high-risk cardiac procedures, are at higher risk for hospital readmissions or death due to inadequate monitoring at home. Research has shown that patients receiving usual care were three times more likely to be readmitted or die following surgery than patients receiving home visits for monitoring and treatment. There is a need for reliable and safe technologies that also provide easy and reliable home monitoring during this 30-day post-discharge, or until the patient's health has stabilized after discharge.

Wearable technology in healthcare includes electronic devices that consumers can wear, like Fitbits fitness tracking devices and smartwatches, and are designed to collect the data of users' personal health and exercise. Biosensors are up and coming wearable medical devices that are radically different from wrist trackers (like Fitbits) and smartwatches. These wearable biosensors are self-adhesive patches that allow patients to move around while collecting data on their movement, heart rate, respiratory rate, temperature, lead 1-ECG and blood pressure. Unfortunately, these solutions have not demonstrated reduction in readmission or death of any causes after discharge. Different methods considered for remote monitoring, focusing on Lead 1-ECGs, weight, intrathoracic impendence, have not been successful in reducing hospitalization rates. In comparison, heart failure management guided by daily measurement of pulmonary pressures has demonstrated substantial reduction in hospitalization rates, linked with reducing pulmonary pressures with diuretic agents and guideline-directed pharmacological therapies. These benefits persist over the full duration of randomized follow-up among general-use patients who had the St. Jude Medical's CardioMEMS pulmonary artery pressure monitoring system implanted since FDA approval and in patients with both preserved and reduced ejection fraction. These observations support the clinical effectiveness of ambulatory pulmonary pressures monitoring as a successful strategy for better management from home.

In light of the problem noted above and breakthrough success of the implanted pulmonary artery pressure monitoring system, the embodiments herein further provide the first non-invasive, battery-less, wearable biosensor for pulmonary artery pressures. The biosensor can be a patch biosensor for short-term hemodynamic monitoring by frequent measurements of pulmonary artery pressure which can also further use HEMOTAG technology developed by AventuSoft based on cardiac time intervals, namely, Isovolumic Contraction Time, Aortic Opening Time, and Systolic Time Ratio. This application of the above cardiac time intervals has been validated by Aventusoft with the gold standard assessment from heart catherization, transthoracic echocardiogram and N-terminal pro b-type natriuretic peptide (NTproBNP) tests. With the need to enable functional independence of adults with chronic heart and lung conditions, a tool for providing non-invasive assessment of hemodynamics is essential in today's age.

Although the embodiments are not limited to the use of a single patch biosensor, there is a great value proposition using the form factor of a Near-Field Communication (NFC) based one-patch biosensor and mobile phone reader. NFC is a wireless communication technology for data transfer between two devices in close proximity to one another (less than 20 cm), and it is more secure than other wireless technologies like Bluetooth and Wi-Fi, with a maximum data transfer rate of 424 kbit/s. NFC-based communication involves an NFC-enabled patch and an NFC-enabled reader. The reader generates a radio frequency field in the 13.56 MHz frequency that can communicate to the patch which can power itself from the radio-frequency waves generated by the reader. In this passive mode, the patch will be powered only when it is in the vicinity of an active NFC reader. This flexibility allows the passive NFC patch to take a very light-weight, simple and miniature form factor, and can be worn for extended periods of time where the typical usage would likely involve frequent measurements for monitoring. As there is no battery requiring charging for the patch, a detachment and reattachment of the patch would be obviated and essentially eliminated for such purpose.

Furthermore, as part of the value proposition, there is a great installed base of NFC readers. NFC readers are built into almost all mobile smartphones and smartwatches today, making it extremely convenient for patients. NFC enabled patch solutions enable on-need assessment of physiological parameters without tethering the patient to a wired hub or requiring device placement every time or requiring its battery to be charged every so often. In medical devices domain, the need for a secure communication channel cannot be over-stressed, and it is an overriding priority. Hence, NFC is gaining traction as the natural choice for wirelessly communicating between two medical devices.

The one-patch design of the system means a single connection point and easier use of the device, compared with a multi-patch design, and provides a more reliable connection with reduced noise and error in measurement due to an unstable connection or a deviation from a nominal position, both due to the risk of user inability to successfully mount a multi-patch device with multiple points of contact. The battery-less design of the system makes it over 80% lighter, over 50% slimmer, and over 75% smaller than a comparable battery-powered sensor. The lighter and smaller design allows for longer duration of wear and minimal obstruction with clothing and daily activities, mitigating the risk of device displacement and corresponding reading(s) error. The one patch form factor also makes it easier to compare and follow consequent readings during patient monitoring. As the patch does not need re-attachment, it significantly reduces skin abrasion associated from repeated application of ECG electrode patches.

The system uses an algorithm for heart and lung function assessment using a short snapshot of NFC patch biosensor. Data assessment based on single-channel localized readings supports the patch biosensor, which makes device attachment and detachment easier compared with other devices that require reading from different locations (channels) far enough to be integrated into a single patch. The algorithm can provide complex hemodynamic parameters from a short snapshot of 10 seconds of, 1-lead ECG and 1-channel accelerometer or cardiac waveform data captured via the patch, which supports the frequent usage of the device and is pertinent to the NFC data transfer rate requirements.

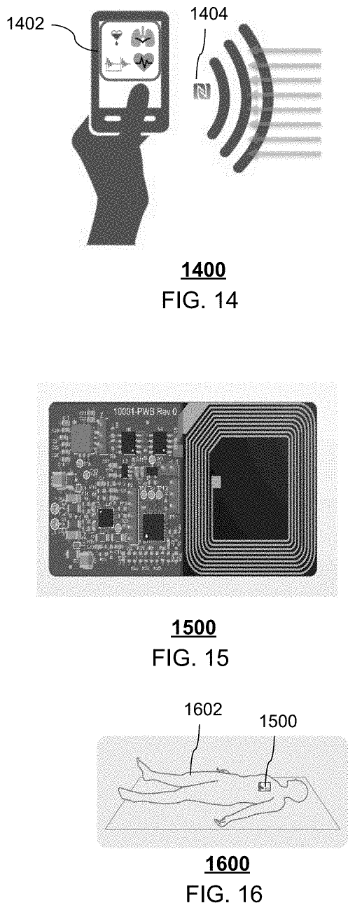

Referring to FIG. 14, a system 1400 includes a smartphone 1402 such as an iOS or Android smartphone to read cardiac data from the biosensor 1404 using the built-in Near-field Communication (NFC) and to the display physiological data. FIG. 15 illustrates a non-invasive, battery less NFC based biosensor sticker 1500 that can capture 10 seconds of, 1-lead ECG and 1-channel accelerometer or cardiac waveform data. Referring to the system 1600 of FIG. 16, the biosensor 1500 can be placed anywhere on the sternum of a patient 1602. For recording a test, the patient while lying down places the mobile phone (see FIG. 14) on the chest, with the biosensor touching the skin. After 10 seconds of data collection, the hemodynamic assessment measurements of heart rate, breathing rate, 1-lead ECG, 1 channel heart sound strip are ready for display on the phone and transmission to a {HIPAA-compliant secured and protected health information or PHI} cloud dashboard for remote monitoring. For example, Microsoft Azure Cloud Services can be used with further following HIPAA regulations by entering into contracts (BAAs) with Microsoft to ensure that the cloud provider will adequately protect PHI.

Again, a system as shown in FIGS. 14-16 can include a non-invasive cardiac pressure sensor and delivery system which can include a wireless, non-invasive, battery-less, disposable (typically one-patch) cardiac biosensor providing hemodynamic measurements proven in reducing hospitalization rates and better management from home. This innovation extracts events related to heart valve openings and closings from a single channel/location on the chest, to provide an on-demand assessment of hemodynamics leveraging the benefits of NFC. The design in FIG. 15 serves as a prototype for an NFC biosensor. The biosensor design incorporates a combination of sensing technologies which enable 1-lead ECG measurement and 1 microelectromechanical systems (MEMS) accelerometer for capturing cardiac vibrations which ideally can be on a short snapshot of 10 seconds. The biosensor handles the signal conditioning, processes the data, enables data logging of the data via on-board ferroelectric random-access memory and provides the radio frequency communication link through near field communication (NFC) to an available reader, in this case an iOS or Android NFC enabled mobile phone. (Of course, other NFC enabled readers can be used in the alternative). Without a battery, the biosensor is functional when in proximity of the reader, scavenging the radio frequency energy of the radiating element through inductive coupling. As shown in FIG. 14, a smartphone app can trigger the data read from the biosensor when brought in its proximity. In some embodiments, a new biosensor as contemplated would be useful for in-depth hemodynamic monitoring beyond simple heartbeat detection, providing, hemodynamic assessment of at least one or more of the following—1) Right Ventricular Systolic Pressure (RVSP), 2) mean Pulmonary Artery Pressure (mPAP), 3) Left ventricular end-diastolic pressure (LVEDP), 4) Ejection fraction, 5) Left Atrial Volume Index.

In some embodiments, the biosensor also provides 1-lead ECG, along with the assessment of heart rate, breathing rate, atrial fibrillation, tachycardia, bradycardia. The hemodynamic measurements are available instantly on the smartphone application, and can further transmit such data to a HIPAA compliant secure dashboard for sharing with the patients' primary and specialist healthcare providers. This provides a reliable, robust, simple, wearable, and portable solution with no need for detachment and reattachment on frequent measurements over time so that the individuals being monitored are engaged and empowered in their own healthcare.

The proposed project has the potential to advance scientific knowledge and available technological capability by improving care while reducing diagnostic errors. This technology is not just a minor improvement over existing technologies, but a major step up from current technology that does not exist today.

Even with all the advances in medical science, there is no simple non-invasive portable method for accurately measuring patient cardiac pressures. Available instruments have their own specialties: lead 1-ECG (arrhythmias), stethoscope (murmurs), portable echocardiogram (wall-motion abnormalities), pulse oximeter (oxygenation), blood pressure cuff (hypertension), and scale (weight gain). Yet for cardiac pressure, doctors still use patient signs, symptoms, and response-to-medication to make a diagnosis. Instrumental evaluation, where available, is an echocardiogram (that is specialized) or a catheter (that is invasive). This can be too late for some patients as noted above. Accessing cardiovascular signals recorded by an easy-to-use noninvasive monitoring device, that enables frequent measurements in a short course of time, can provide unique insights into heart failure pathophysiology as patients progress from stable to congested states.

Tracking the filling pressure using hemodynamic monitoring can reveal valuable information of an upcoming congestion and hospitalization ahead of time before symptom appear, when it might be too late for some patients.

The small portable wireless device is easy to use by a patient, providing rapid accurate assessment with at-home monitoring capability, when it's needed, where it's needed, for faster diagnosis and treatment and for monitoring purposes. The device provides absolute measures without requiring invasive implants or invasive tests. The device and services can be scaled rapidly in the field due to its operation via a central (HIPAA-compliant) cloud system, along with an easy-to-use hardware.

The desirability of mobile health solution disclosed herein is two-fold for its cost savings and availability.

With respect to cost savings, depending on where a patient undergoes an echocardiogram, the test can cost somewhere between $100 and $1,000. Close to 8 million echocardiograms and 1 million cardiac catheterizations are performed in the U.S. annually. In 2010, echocardiogram testing alone accounted for over $1.1 billion, or nearly 11 percent, of the amount Medicare spent in total imaging costs that year. Researchers argue that the public health system could have saved millions of dollars if there were alternatives. Patients who receive echocardiography tests, most receive approximately one test a year. Patients living in higher population areas are more likely to have a test repeated. Echocardiogram and catheterization provides a comprehensive evaluation of the cardiac function of patient, but such a comprehensive evaluation is unnecessary to identify or track ventricular dysfunction in a patient. The mobile health solution has the advantage of providing the specific pressure assessment, and can be provided at a fraction of the cost, and can be carried out by the patient for an extended period of time from home.

With respect to availability, rates of echocardiography and invasive diagnostic imaging are lower in the Northern U.S. and higher in the Southeastern U.S. Use of diagnostic imaging is higher in urban areas than for patients in rural areas. This may be due to greater distance for those in rural areas to facilities offering echocardiography and catheterization, suggesting that greater patient volume may be associated with the decision to invest in diagnostic imaging equipment. The mobile health solution with a cost much cheaper to that of an electronic stethoscope, can be used by any physician for diagnosis and patient management. Thus, providing improved quality of care, especially in underserved areas.

Note that patients are now routinely released on their fourth day, thereby making post monitoring from home ever more essential. Patients discharged following heart failure, myocardial infarction, coronary artery bypass grafting surgery or other high-risk cardiac procedures, are at higher risk for hospital readmissions or death due to inadequate monitoring at home. Research has shown that patients receiving usual care were three times more likely to be readmitted or die following surgery than patients receiving home visits for monitoring and treatment. This indicates a great need for home monitoring.

This technology can measure LVEDP to non-invasively diagnose Heart failure, both with Reduced and Preserved ejection fraction and has a number of advantages over other non-invasive technologies such as, digital stethoscope and portable ECG-based devices (such as EKO device, AliveCor's KardiaMobile), and wearables including FitBit, Apple Watch, and others. The other device do not provide hemodynamic measurements necessary for heart failure management and in fact, such other device have warnings or U.S. FDA Indications for Use caution “DO NOT use to diagnose heart related conditions”. New investigational technologies under development that use ECG with arterial pressure waveform or use finger photo plethysmography during Valsalva have limitations of requiring the invasively obtained arterial pressure waveform or the lack of an absolute hemodynamic marker of pressure respectively.

The mobile health solution presented will be an industry first to provide accurate cardiac pressure assessment non-invasively and report wirelessly via any smartphone anywhere, anytime, by anyone. The framework can use a specially engineered accelerometer transducer fused with an electrocardiogram transducer system and biologically-inspired algorithms that are accessible via a smartphone device for a portable and cost-effective tool. The proposed device fits an important critical medical need and provides a significant advantage due to its following features: 1) It will allow direct non-invasive measurement of a patient's cardiac function; 2) It will provide an easy-to-read visual display; 3) The indicators will enable a physician to use the standard of care in treating a patient; 4) The device will enable healthcare professionals to administer the data collection; 5) The novel sensor framework uses an electrocardiogram sensor electrode that provides the necessary signal capture while minimizing noise and the effects of patient variability; and 6) It will include ECG measurement. The NFC one-patch and battery-less design gives it technical advantages that provide revolutionary vital sign monitoring and make it a foundation for other useful devices.

In some embodiments, this Mobile health solution can be used for up to 30 days (used as an initial evaluation period (extendable to 60 days or 90 days or possibly longer). In patients with chronic heart problems, the patient can be included in a Remote Physiological Monitoring (RPM) program. The data can be sent to a HIPAA compliant cloud monitoring system and interpreted by clinical staff, after which the clinical staff can call the care provider and the patient for treatment managements.

RPM is shown to be very effective in tackling high, rising readmission rates for congestive heart failure. RPM not only reduces the readmission rate and all-cause mortality but also is cost-effective. With all these benefits, RPM is not a replacement for primary care visits (PCV), instead it is an effective follow up method that provides patients the advantages of 30 days monitoring that can extend to 90 days. Providing such a service is not doable using PCV.

In some embodiments, the system uses the application of Cardiac Time Intervals (CTIs) to estimate absolute hemodynamics. The Cardiac Time Intervals (CTIs) are defined by the opening and closing of the valves of the heart with respect to the start of the QRS complex. In prospective studies, CTIs have been shown to be predictive of hemodynamics because they are indicative of myocardial structure and function. For example, Weissler and colleagues demonstrated a non-invasively measured index for systolic function, Systolic Time Ratio (STR=PEP/LVET), defined as the pre-ejection period (PEP=start of Q of ECG to opening of the Aortic valve) divided by the Left Ventricular Ejection Time (LVET=Opening of Aortic valve to closing of the Aortic valve). Multiple large studies have validated the relationship and accuracy between the cardiac time intervals and systolic and diastolic function assessed with conventional, tissue Doppler, and speckle-tracking echocardiography. The prognostic value of cardiac time intervals has previously been shown in various populations, e.g., following acute myocardial infarction, in elderly men, in American Indians, the general population, patients with cardiac amyloidosis, in patients with idiopathic-dilated cardiomyopathy, in isolated diastolic dysfunction, in patients with systolic heart failure, and to guide outpatient therapy of patients with acute Heart Failure syndrome.

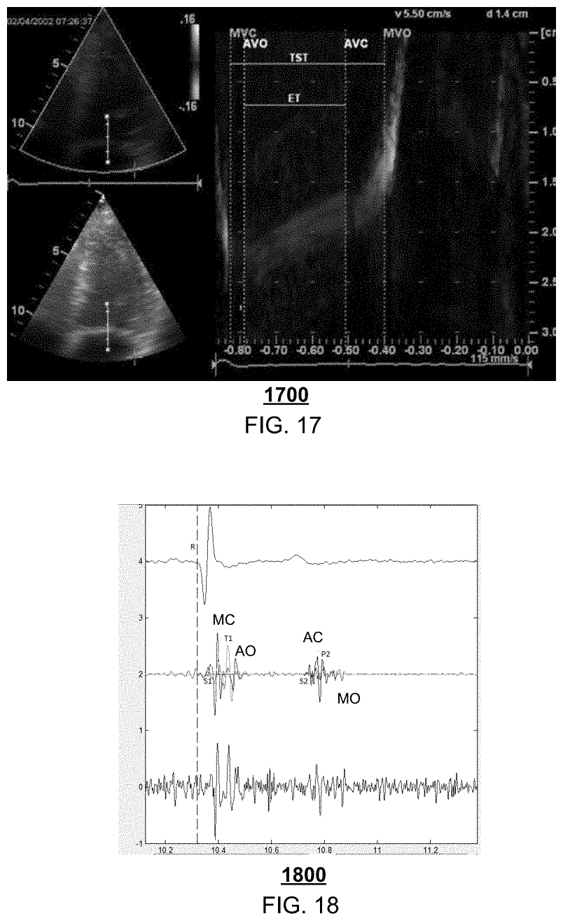

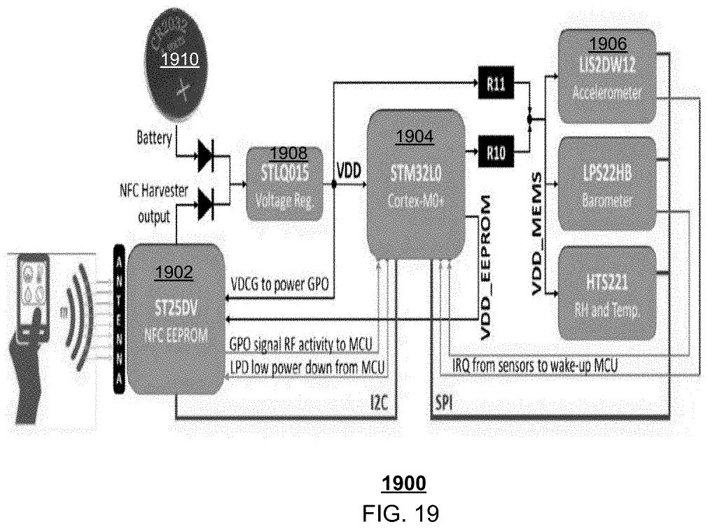

The chart 1700 in FIG. 17 illustrates CTIs assessed by a tissue Doppler imaging. The chart 1800 illustrates Aventusoft's algorithm for CTI measurement in accordance with the embodiments. Such a system can provide simultaneous ECG and heart vibration recording with annotations—MC: Mitral valve closure; AO: Atrial valve opening; AC: Aortic valve closure; MO: Mitral valve opening. In other embodiments, additional annotations can include PEP: Pre-ejection period; LVET: Left ventricular ejection time; IVCT: Isovolumetric contraction time; IVRT: Isovolumetric relaxation time.

Currently CTI based hemodynamics can be obtained by the conventional echocardiogram, using the regional TDI velocity curves or by TDI M-mode. Alternatively, the measurement of hemodynamics is only possible with a heart catheterization or a transthoracic echocardiogram. Hemodynamic measurements are not possible with a digital stethoscope, portable ECG, heart rate wearables, or a hand-held Echocardiogram. Identifying the accurate time of color change manually by the sonographer is prone to errors and can result in poor estimation of valve openings (as shown in FIG. 17). Aventusoft's algorithms extract events related to heart valve openings-closings from composite heart vibrations captured from multiple channels/locations on the chest. This new innovation extracts events related to heart valve openings-closings from a single channel and location on the chest. The technology separates the Mitral and Aortic valve events. The performance of the technology is shown in the chart 1800 FIG. 18. This capability allows the algorithm to provide the hemodynamic measurements of, 1) left ventricular systolic function, 2) left ventricular diastolic function, and 3) pulmonary artery pressures. From a 30 second multi-channel heart sound data, the algorithm can also detect abnormal heart rhythms (arrhythmias), uncoordinated contractions (fibrillation), and intermittent pacing. It eliminates the abnormal beats and accommodates for any heart rate changes, to provide an accurate calculation of the cardiac time intervals. This allows it to operate successfully in acquiring and analyzing data in patients with frequent arrhythmias and/or intermittent pacing, which has been validated through a WIRB approved clinical study.

In some embodiments, the system can use a HEMOTAG cardiopulmonary assessment system (CPAS). HEMOTAG CPAS in a different form factor uses three highly sensitive low-noise accelerometer sensors to capture micro vibrations on the chest along with 3 adhesive gel-based ECG electrodes providing 1-lead ECG signal, to provide the estimate of Ejection Fraction. Our newer NFC based cardiac biosensor uses the same highly sensitive low-noise single accelerometer and uses two dry electrodes to provide 1-lead ECG, making it extremely compact, battery-less and without requiring disposable gel ECG electrodes.

The HEMOTAG based hemodynamic measurements can be used to accurately detect acute decompensation of heart failure (ADHF) and improvement following treatment. Aventusoft completed a clinical study where patients underwent NTproBNP measurement and HEMOTAG examination at day 1 and day 3 for HEMOTAG based cardiac time intervals, isovolumetric contraction time (IVCT). The study successfully validated HEMOTAG based hemodynamic measurements to accurately detect acute decompensation of heart failure and improvement following treatment. This hemodynamic assessment using IVCT will be provided by the biosensor developed in this innovation.

The study demonstrated HEMOTAG can be successfully used for diagnosis and monitoring patients with the following 15 disease states: Fluid-management problems, HF, coronary artery disease, hypertension, pulmonary hypertension, DM, Lung Disease, mitral valve Disease, aortic valve Disease, Ischemic Cardiomyopathy, LV Hypertrophy, NYHA HF, Sleep Apnea, Angina, and previous Myocardial Infarction.

The mobile health system includes some advantages and innovations over the current 3 channel form factor for HEMOTAG. The HEMOTAG device records 30 seconds of, 1-lead ECG and 3-channel accelerometer or cardiac waveform data. HEMOTAG electrodes are intendedly designed to be apart to record signals from different places of the chest to provide a rich profile of heart valvular activity with partially redundant information that is essential to that hemodynamic assessment algorithm. The lengthy data recording (30 seconds) is also required to mitigate noise and intra-subject variability that could otherwise affect the performance of the HEMOTAG. Furthermore, HEMOTAG is battery-powered consistent with its desired usage of less-frequent standalone measurements over long-term. In contrast, the mobile health system is based on a different algorithm with easier requirements that enables a different design. It comes with a quite localized biosensor integrated into a single patch, which makes it a more patient-friendly wearable; an important factor, particularly if used at home and by the patient or a patient assistant. The mobile health system's recording time is also shorter and has a single accelerometer or cardiac waveform sensor (10 seconds of, 1-lead ECG and 1-channel accelerometer or cardiac waveform data), which suits the NFC maximum data transfer rate requirement.

Development efforts for implementing the NFC sensor that includes the novel non-invasive, battery-less, cardiac biosensor for capturing the cardiac vibrations which can be converted into cardiac time intervals initially used and evaluated two reference designs for the NFC biosensor. A proposed development of a commercial version of the mobile health solution can include the following components: 1) the battery-less biosensor, and 2) A mobile application for measuring and reporting hemodynamics, heart-rate, breathing rate, 1-lead ECG, and heart sound waveform.

In some embodiments, a commercial system for the sensor system would include 1) NFC based battery-less, disposable cardiac biosensor, 2) an Algorithm for heart and lung function assessment using 10 seconds of 1-lead ECG and 1-channel accelerometer or cardiac waveform data, which would further include 3) a Human factor design and validation user study, and 4) Testing and FDA 510(k) clearance.

In some embodiments, the system can provide simultaneous ECG and heart vibration recording with annotations—MC: Mitral valve closure; AO: Atrial valve opening; AC: Aortic valve closure; MO: Mitral valve opening; PEP: Pre-ejection period; LVET: Left ventricular ejection time; IVCT: Isovolumetric contraction time; IVRT: Isovolumetric relaxation time. In some embodiments, the system can identify the cardiac time-intervals indicative of hemodynamic changes, using 10-second data from a single accelerometer and ECG signal. Annotated signals from a single sensor with a synchronous ECG signal can be presented. The challenge addressed through this aim is to develop an algorithm for accurately identifying these heart valves closing and opening events captured from the NFC biosensor using a short snap shot of data recorded using a single channel accelerometer data where ideally a powerful algorithm does not rely on redundant input to tackle and tolerate input noise and intra-subject variability. Some embodiments can provide a beat-to-beat assessment of cardiac time intervals by correct identification of peaks and valleys associated with aortic valve opening/closing, mitral valve opening/closing, by automated localization of valve events with deep learning using convolutional neural network. The inputs are the padded 400 data-points cardiac complexes. The last layer is the output layer with 4 neurons. Each of these 4 neurons predicts the location of each Mitral closing, Aortic opening, Aortic closing and Mitral opening. Mean Root Square Error (MRSE) is used as the loss function. We have synchronous ECG and annotated heart-valves data available from over 400+ patients 30-second recordings; through our past clinical trials, this provides an adequate database for training the deep network. Thus, the mobile health solution's algorithms will have the unique advantage of providing the mitral and aortic valve opening and closings events, enabling the measurement of time intervals (AVO, IVCT, STR). By the end of this specific aim, the algorithm will provide a measure of patient's heart function—providing estimates of left ventricular systolic function, left ventricular diastolic function, any tachycardia, bradycardia, uncoordinated contractions, heart rate. The algorithm will measure the patient's lung function—providing estimates of any elevation in pulmonary artery pressure indicating pulmonary congestion.

In some embodiments, the system include a Mobile App that can be downloaded to the patient's smartphone and made available for both Apple iOS-based and Android-based smartphones. The biosensor connects wirelessly to the app, enabling the patient to start registration and follow instructions for measurement. A PDF with the ECG from the measurement is generated and can be viewed and saved on the patient's phone. Immediately after the measurement is completed, the patient can view the automatically analyzed results. The Mobile App also includes “My journal,” where the results are then stored. The development will leverage the base application available from the NFC manufacturer to provide the NFC communication framework. Our current HEMOTAG mobile applications for iOS and Android will be modified to work the NFC biosensor.

The biggest challenge is the use of 10 seconds of data from a single-channel accelerometer sensor of the mobile health solution herein instead of 30 seconds data from three channels of accelerometer of HEMOTAG. To tackle this problem we design and implement an algorithm with easier assumptions on input data: the multiple-input data is not required and works beat-to-beat. The identified cardiac events will be compared against reference HEMOTAG recordings, which is already validated by the gold standard echocardiogram.

It will be apparent to those skilled in the art that various modifications may be made in the present invention without departing from the spirit or scope of the invention. Thus, it is intended that the present invention cover the modifications and variations of this invention provided they come within the scope of the method and system described and their equivalents.

What is claimed is:

1. A health sensor system, comprising:
a wearable non-invasive biosensor for capturing one or more cardiac waveform signals using one or more noninvasive sensors or transducers over one or more channels where near field communication (NFC) is used to power the wearable non-invasive biosensor and to communicate between the wearable non-invasive biosensor and health sensor system;
one or more processors operatively coupled to the wearable non-invasive biosensor;
memory having computer instructions and coupled to the one or more processors, the computer instructions which when executed by the one or more processors causes the system to perform the operations of:
powering the health sensor system in response to receiving a radio frequency signal;
monitoring cardiac pressures and cardiac function based on cardiac time intervals during a period when the health sensor system is powered by the radio frequency signal;
performing a heart and lung function assessment based on the monitoring of the cardiac pressures; and
presenting the heart and lung function assessment.

2. The health sensor system of claim 1, wherein the wearable non-invasive biosensor is a patch biosensor.

3. The health sensor system of claim 1, wherein the wearable non-invasive biosensor is a single near-field communication patch biosensor.

4. The health sensor system of claim 1, wherein the cardiac time intervals are measurements of at least one of isovolumic contraction time, aortic opening time, aortic closing time, mitral opening time, mitral closing time, and systolic time ratio.

5. The health sensor system of claim 1, wherein the wearable non-invasive biosensor uses a 1-lead ECG sensor and a single channel cardiac waveform sensor or accelerometer to provide data supporting frequent usage at a data rate compatible with the near field communication protocol.

6. The health sensor system of claim 1, wherein the wearable non-invasive biosensor is a patch sensor that frequently captures no more than 10 seconds of 1-lead ECG data and a single channel cardiac waveform signal or accelerometer data to provide data supporting frequent usage at a data rate compatible with the near field communication protocol.

7. The health sensor system of claim 1, wherein the wearable non-invasive biosensor is a patch sensor that frequently captures a predetermined time period of data to perform hemodynamic assessment measurements of blood oxygen level measurement, heart rate, breathing rate, arrhythmias, fibrillations, 1-lead ECG rhythm data, 1 channel of heart vibration data.

8. The health sensor system of claim 7, wherein presenting the heart and lung function and pressure assessment comprises displaying the heart and lung function assessment on a phone in close proximity to the patch sensor.

9. The health sensor system of claim 7, wherein presenting the heart and lung function and pressure assessment comprises displaying, analyzing and monitoring the heart and lung function assessment on a remote monitoring station and optionally alerting for health status conditions.

10. The health sensor system of claim 7, wherein the one or more processors further perform the operation of securely transmitting via a HIPAA-compliant network for remote monitoring of the hemodynamic assessment measurements.

11. The health sensor system of claim 1, wherein the wearable non-invasive biosensor is a patch sensor that frequently captures a predetermined time period of data to perform hemodynamic assessment measurements of one or more cardiac pressures among others Right Ventricular Systolic Pressure (RVSP), mean Pulmonary Artery Pressure (mPAP), Left ventricular end-diastolic pressure (LVEDP), and cardiac function among others E/e′, Ejection fraction or Left Atrial Volume Index.

12. The health sensor system of claim 1, wherein the one or more processors further perform the operation of estimating absolute hemodynamics using cardiac time intervals defined by opening and closing of valves of a heart with respect to a start of a QRS complex as measured by the wearable non-invasive biosensor capturing ECG signals.

13. A health sensor system, comprising:
a wearable non-invasive biosensor in the form of a single near-field communications (NFC) patch biosensor configured for capturing one or more cardiac waveform signals over one or more channels and configured to use near field communications to power the NFC patch biosensor and to communicate between the NFC patch biosensor and an NFC reader;
one or more processors operatively coupled to the wearable non-invasive biosensor;
memory having computer instructions and coupled to the one or more processors, the computer instructions which when executed by the one or more processors causes the system to perform the operations of:
powering the health sensor system in response to receiving a radio frequency signal;
monitoring pulmonary artery pressures based on cardiac time intervals during a period when the health sensor system is powered by the radio frequency signal;
performing a heart and lung function assessment based on the monitoring of the pulmonary artery pressures; and
presenting the heart and lung function assessment.

14. The health sensor system of claim 13, wherein the cardiac time intervals are measurements of isovolumic contraction time, aortic opening time, aortic closing time, mitral opening time, mitral closing time, and systolic time ratio.

15. The health sensor system of claim 13, wherein the wearable non-invasive biosensor uses a 1-lead ECG sensor and a single channel accelerometer or cardiac waveform sensor to provide data supporting frequent usage at a data rate compatible with the near field communication protocol.

16. The health sensor system of claim 13, wherein the wearable non-invasive biosensor frequently captures no more than 10 seconds of 1-lead ECG data and a single channel of accelerometer data or a single channel of cardiac waveform data to provide data supporting frequent usage at a data rate compatible with the near field communication protocol.

17. The health sensor system of claim 13, wherein the wearable non-invasive biosensor frequently captures a predetermined time period of data to perform hemodynamic assessment measurements of one or more cardiac pressures among others Right Ventricular Systolic Pressure (RVSP), mean Pulmonary Artery Pressure (mPAP), Left ventricular end-diastolic pressure (LVEDP), cardiac function among others E/e′, Ejection fraction or Left Atrial Volume Index. (ratio of ejection fraction?? E to e′ ratio to evaluate the LVFP)

18. The health sensor system of claim 13, wherein the one or more processors further perform the operation of estimating absolute hemodynamics using cardiac time intervals defined by opening and closing of valves of a heart with respect to a start of a QRS complex as measured by the wearable non-invasive biosensor capturing ECG signals.

19. A method of providing a heart and lung function assessment using a wearable non-invasive biosensor, the method comprising:
capturing electrocardiogram (ECG) signals and composite vibration objects over one or more channels using the non-invasive biosensor in the form of a single near-field communications patch biosensor;
powering the non-invasive biosensor in response to receiving a radio frequency signal using a near field communication protocol;
monitoring pulmonary artery pressures based on cardiac time intervals during a period when the health sensor system is powered by the radio frequency signal;
performing a heart and lung function assessment based on the monitoring of the pulmonary artery pressures; and
presenting the heart and lung function assessment.

20. The method of claim 19, wherein the method further comprises transmitting the captured ECG signals and composite vibration objects or cardiac time intervals to an NFC reader and wherein the cardiac time intervals are measurements of isovolumic contraction time, aortic opening time, aortic closing time, mitral closing time mitral opening time, and systolic time ratio.

21. The method of claim 19, wherein the step of performing the heart and lung function assessment includes the monitoring and indirectly tracking of filling pressures over a time period using multiple recordings to recognize disease states and to anticipate upcoming congestion conditions before symptoms appear for cardiac patients.

22. The method of claim 19, wherein performing the heart and lung function assessment includes early detection or anticipatory detection of Coronary artery disease, Heart murmurs, valve abnormalities, Heart failure, Heart rhythm abnormalities (arrhythmias), Vascular disease, congenital heart disease, Cardiac resynchronization and cardiac risk factors.
